SEQUENCE LISTING (25 sequences)

SEQ ID NO: 1 - skipped

SEQ ID NO: 2 - protein, 38 aa, synthetic construct
  VARIANT 4: note = X is an aromatic amino acid residue, a hydrophobic amino acid residue, or a neutral amino acid residue
  VARIANT 7: note = X is an aromatic amino acid residue, a hydrophobic amino acid residue, or a neutral amino acid residue
  VARIANT 11: note = X is an aromatic amino acid residue, a hydrophobic amino acid residue, or a neutral amino acid residue
  VARIANT 14: note = X is an aromatic amino acid residue, a hydrophobic amino acid residue, or a neutral amino acid residue
  VARIANT 18: note = X is an aromatic amino acid residue, a hydrophobic amino acid residue, or a neutral amino acid residue
  VARIANT 21: note = X is an aromatic amino acid residue, a hydrophobic amino acid residue, or a neutral amino acid residue
  VARIANT 21: note = X is an aromatic amino acid residue, a hydrophobic amino acid residue, or a neutral amino acid residue
  VARIANT 25: note = X is an aromatic amino acid residue, a hydrophobic amino acid residue, or a neutral amino acid residue
  VARIANT 28: note = X is an aromatic amino acid residue, a hydrophobic amino acid residue, or a neutral amino acid residue
  VARIANT 32: note = X is an aromatic amino acid residue, a hydrophobic amino acid residue, or a neutral amino acid residue
  VARIANT 35: note = X is an aromatic amino acid residue, a hydrophobic amino acid residue, or a neutral amino acid residue
VKEXTFXKNTXPQXLREXQEXNAAXQDXRELXRQXSKL

SEQ ID NO: 3 - protein, 38 aa, synthetic construct
  VARIANT 4: note = X is are each independently an aromatic amino acid residue, a hydrophobic amino acid residue, or a neutral amino acid residue
  VARIANT 7: note = X is are each independently an aromatic amino acid residue, a hydrophobic amino acid residue, or a neutral amino acid residue
  VARIANT 11: note = X is are each independently an aromatic amino acid residue, a hydrophobic amino acid residue, or a neutral amino acid residue
  VARIANT 14: note = X is are each independently an aromatic amino acid residue, a hydrophobic amino acid residue, or a neutral amino acid residue
  VARIANT 18: note = X is are each independently an aromatic amino acid residue, a hydrophobic amino acid residue, or a neutral amino acid residue
  VARIANT 21: note = X is are each independently an aromatic amino acid residue, a hydrophobic amino acid residue, or a neutral amino acid residue
  VARIANT 25: note = X is are each independently an aromatic amino acid residue, a hydrophobic amino acid residue, or a neutral amino acid residue
  VARIANT 28: note = X is are each independently an aromatic amino acid residue, a hydrophobic amino acid residue, or a neutral amino acid residue
  VARIANT 32: note = X is are each independently an aromatic amino acid residue, a hydrophobic amino acid residue, or a neutral amino acid residue
  VARIANT 35: note = X is are each independently an aromatic amino acid residue, a hydrophobic amino acid residue, or a neutral amino acid residue
VKEXLFXKKTXEQXLEEXKEXNKAXHDXRHLXENXSKL

SEQ ID NO: 4 - protein, 38 aa, synthetic construct
  VARIANT 4: note = X is an aromatic amino acid residue, a hydrophobic amino acid residue, or a neutral amino acid residue
  VARIANT 7: note = X is an aromatic amino acid residue, a hydrophobic amino acid residue, or a neutral amino acid residue
  VARIANT 11: note = X is an aromatic amino acid residue, a hydrophobic amino acid residue, or a neutral amino acid residue
  VARIANT 14: note = X is an aromatic amino acid residue, a hydrophobic amino acid residue, or a neutral amino acid residue
  VARIANT 18: note = X is an aromatic amino acid residue, a hydrophobic amino acid residue, or a neutral amino acid residue
  VARIANT 21: note = X is an aromatic amino acid residue, a hydrophobic amino acid residue, or a neutral amino acid residue
  VARIANT 25: note = X is an aromatic amino acid residue, a hydrophobic amino acid residue, or a neutral amino acid residue
  VARIANT 28: note = X is an aromatic amino acid residue, a hydrophobic amino acid residue, or a neutral amino acid residue
  VARIANT 32: note = X is an aromatic amino acid residue, a hydrophobic amino acid residue, or a neutral amino acid residue
  VARIANT 35: note = X is an aromatic amino acid residue, a hydrophobic amino acid residue, or a neutral amino acid residue
VKEXLFXKNTXYQXLLEXKEXNEAXYDXRHLXQQXSKL

SEQ ID NO: 5 - protein, 38 aa, synthetic construct
  VARIANT 4: note = X is an aromatic amino acid residue, a hydrophobic amino acid residue, or a neutral amino acid residue
  VARIANT 7: note = X is an aromatic amino acid residue, a hydrophobic amino acid residue, or a neutral amino acid residue
  VARIANT 11: note = X is an aromatic amino acid residue, a hydrophobic amino acid residue, or a neutral amino acid residue
  VARIANT 14: note = X is an aromatic amino acid residue, a hydrophobic amino acid residue, or a neutral amino acid residue
  VARIANT 18: note = X is an aromatic amino acid residue, a hydrophobic amino acid residue, or a neutral amino acid residue
  VARIANT 21: note = X is an aromatic amino acid residue, a hydrophobic amino acid residue, or a neutral amino acid residue
  VARIANT 25: note = X is an aromatic amino acid residue, a hydrophobic amino acid residue, or a neutral amino acid residue
  VARIANT 28: note = X is an aromatic amino acid residue, a hydrophobic amino acid residue, or a neutral amino acid residue
  VARIANT 32: note = X is an aromatic amino acid residue, a hydrophobic amino acid residue, or a neutral amino acid residue
  VARIANT 35: note = X is an aromatic amino acid residue, a hydrophobic amino acid residue, or a neutral amino acid residue
VKEXTFXKKTXEQXAEEXKEXNKAXHDXRHAXENXSKK

SEQ ID NO: 6 - protein, 38 aa, synthetic construct
  VARIANT 4: note = X is an aromatic amino acid residue, a hydrophobic amino acid residue, or a neutral amino acid residue
  VARIANT 7: note = X is an aromatic amino acid residue, a hydrophobic amino acid residue, or a neutral amino acid residue
  VARIANT 11: note = X is an aromatic amino acid residue, a hydrophobic amino acid residue, or a neutral amino acid residue
  VARIANT 14: note = X is an aromatic amino acid residue, a hydrophobic amino acid residue, or a neutral amino acid residue
  VARIANT 18: note = X is an aromatic amino acid residue, a hydrophobic amino acid residue, or a neutral amino acid residue
  VARIANT 21: note = X is an aromatic amino acid residue, a hydrophobic amino acid residue, or a neutral amino acid residue
  VARIANT 25: note = X is an aromatic amino acid residue, a hydrophobic amino acid residue, or a neutral amino acid residue
  VARIANT 28: note = X is an aromatic amino acid residue, a hydrophobic amino acid residue, or a neutral amino acid residue
  VARIANT 32: note = X is an aromatic amino acid residue, a hydrophobic amino acid residue, or a neutral amino acid residue
  VARIANT 35: note = X is an aromatic amino acid residue, a hydrophobic amino acid residue, or a neutral amino acid residue
VKEXTFXKNTXEQXAEEXKEXNKAXHDXRHQXENXSKQ

SEQ ID NO: 7 - protein, 38 aa, synthetic construct
  VARIANT 4: note = X is an aromatic amino acid residue, a hydrophobic amino acid residue, or a neutral amino acid residue
  VARIANT 7: note = X is an aromatic amino acid residue, a hydrophobic amino acid residue, or a neutral amino acid residue
  VARIANT 11: note = X is an aromatic amino acid residue, a hydrophobic amino acid residue, or a neutral amino acid residue
  VARIANT 14: note = X is an aromatic amino acid residue, a hydrophobic amino acid residue, or a neutral amino acid residue
  VARIANT 18: note = X is an aromatic amino acid residue, a hydrophobic amino acid residue, or a neutral amino acid residue
  VARIANT 21: note = X is an aromatic amino acid residue, a hydrophobic amino acid residue, or a neutral amino acid residue
  VARIANT 25: note = X is an aromatic amino acid residue, a hydrophobic amino acid residue, or a neutral amino acid residue
  VARIANT 28: note = X is an aromatic amino acid residue, a hydrophobic amino acid residue, or a neutral amino acid residue
  VARIANT 32: note = X is an aromatic amino acid residue, a hydrophobic amino acid residue, or a neutral amino acid residue
  VARIANT 35: note = X is an aromatic amino acid residue, a hydrophobic amino acid residue, or a neutral amino acid residue
VKEXTFXKKQXEQXAEEXKEXNKAXHDXRHAXENXSKK

SEQ ID NO: 8 - protein, 38 aa, synthetic construct; identical to SEQ ID NO: 5
  VARIANT 4: note = X is an aromatic amino acid residue, a hydrophobic amino acid residue, or a neutral amino acid residue
  VARIANT 7: note = X is an aromatic amino acid residue, a hydrophobic amino acid residue, or a neutral amino acid residue
  VARIANT 11: note = X is an aromatic amino acid residue, a hydrophobic amino acid residue, or a neutral amino acid residue
  VARIANT 14: note = X is an aromatic amino acid residue, a hydrophobic amino acid residue, or a neutral amino acid residue
  VARIANT 18: note = X is an aromatic amino acid residue, a hydrophobic amino acid residue, or a neutral amino acid residue
  VARIANT 21: note = X is an aromatic amino acid residue, a hydrophobic amino acid residue, or a neutral amino acid residue
  VARIANT 25: note = X is an aromatic amino acid residue, a hydrophobic amino acid residue, or a neutral amino acid residue
  VARIANT 28: note = X is an aromatic amino acid residue, a hydrophobic amino acid residue, or a neutral amino acid residue
  VARIANT 32: note = X is an aromatic amino acid residue, a hydrophobic amino acid residue, or a neutral amino acid residue
  VARIANT 35: note = X is an aromatic amino acid residue, a hydrophobic amino acid residue, or a neutral amino acid residue

SEQ ID NO: 9 - protein, 38 aa, synthetic construct
  VARIANT 4: note = X is an aromatic amino acid residue, a hydrophobic amino acid residue, or a neutral amino acid residue
  VARIANT 7: note = X is an aromatic amino acid residue, a hydrophobic amino acid residue, or a neutral amino acid residue
  VARIANT 11: note = X is an aromatic amino acid residue, a hydrophobic amino acid residue, or a neutral amino acid residue
  VARIANT 14: note = X is an aromatic amino acid residue, a hydrophobic amino acid residue, or a neutral amino acid residue
  VARIANT 18: note = X is an aromatic amino acid residue, a hydrophobic amino acid residue, or a neutral amino acid residue
  VARIANT 21: note = X is an aromatic amino acid residue, a hydrophobic amino acid residue, or a neutral amino acid residue
  VARIANT 25: note = X is an aromatic amino acid residue, a hydrophobic amino acid residue, or a neutral amino acid residue
  VARIANT 28: note = X is an aromatic amino acid residue, a hydrophobic amino acid residue, or a neutral amino acid residue
  VARIANT 32: note = X is an aromatic amino acid residue, a hydrophobic amino acid residue, or a neutral amino acid residue
  VARIANT 35: note = X is an aromatic amino acid residue, a hydrophobic amino acid residue, or a neutral amino acid residue
VSEXTEXKKTXEHXAKEXKEXNKAXHTXRHAXENXAKN

SEQ ID NO: 10 - protein, 38 aa, synthetic construct
  VARIANT 4: note = X is an aromatic amino acid residue, a hydrophobic amino acid residue, or a neutral amino acid residue
  VARIANT 7: note = X is an aromatic amino acid residue, a hydrophobic amino acid residue, or a neutral amino acid residue
  VARIANT 11: note = X is an aromatic amino acid residue, a hydrophobic amino acid residue, or a neutral amino acid residue
  VARIANT 14: note = X is an aromatic amino acid residue, a hydrophobic amino acid residue, or a neutral amino acid residue
  VARIANT 18: note = X is an aromatic amino acid residue, a hydrophobic amino acid residue, or a neutral amino acid residue
  VARIANT 21: note = X is an aromatic amino acid residue, a hydrophobic amino acid residue, or a neutral amino acid residue
  VARIANT 25: note = X is an aromatic amino acid residue, a hydrophobic amino acid residue, or a neutral amino acid residue
  VARIANT 28: note = X is an aromatic amino acid residue, a hydrophobic amino acid residue, or a neutral amino acid residue
  VARIANT 32: note = X is an aromatic amino acid residue, a hydrophobic amino acid residue, or a neutral amino acid residue
  VARIANT 35: note = X is an aromatic amino acid residue, a hydrophobic amino acid residue, or a neutral amino acid residue
VKEXKFXKNTXPQXLREXQNXNMAXQDXRELXQQXSTL

SEQ ID NO: 11 - protein, 16 aa, synthetic construct
MRGSHHHHHHGSIEGR

SEQ ID NO: 12 - protein, 54 aa, synthetic construct
  VARIANT 18: note = X is A, E, D, R, H, K, Q, N, or S
  VARIANT 19: note = X is A, E, N, or Q
  VARIANT 20: note = X is an aromatic amino acid residue, a hydrophobic amino acid residue, or a neutral amino acid residue
  VARIANT 21: note = X is A, E, R, D, H, I, L, T, K, Q, or N
  VARIANT 22: note = X is A, F, Q, R, K, H, D, S, or E
  VARIANT 23: note = X is an aromatic amino acid residue, a hydrophobic amino acid residue, or a neutral amino acid residue
  VARIANT 24: note = X is A, K, or E
  VARIANT 25: note = X is A, K, E, D, R, H, Q, or N
  VARIANT 26: note = X is A, T, I, L, or Q
  VARIANT 27: note = X is an aromatic amino acid residue, a hydrophobic amino acid residue, or a neutral amino acid residue
  VARIANT 28: note = X is A, E, D, H, P, I, L, K, Y, N, Q, or R
  VARIANT 29: note = X is A, Q, H, E, D, K, R, or N
  VARIANT 30: note = X is an aromatic amino acid residue, a hydrophobic amino acid residue, or a neutral amino acid residue
  VARIANT 31: note = X is A, L, D, E, K, or I
  VARIANT 32: note = X is A, E, D, H, Y, I, L, R, K, Q, or N
  VARIANT 33: note = X is A, E, or Q
  VARIANT 34: note = X is an aromatic amino acid residue, a hydrophobic amino acid residue, or a neutral amino acid residue
  VARIANT 35: note = X is A, K, E, D, K, R, H, N, or Q
  VARIANT 36: note = X is A, N, D, K, R, H, Q, or E
  VARIANT 37: note = X is an aromatic amino acid residue, a hydrophobic amino acid residue, or a neutral amino acid residue
  VARIANT 38: note = X is A, N, or Q
  VARIANT 39: note = X is K, A, E, I, L, M, R, H, D, Q, N, or S
  VARIANT 40: note = X is A, Q, N, I, or L
  VARIANT 41: note = X is an aromatic amino acid residue, a hydrophobic amino acid residue, or a neutral amino acid residue
  VARIANT 42: note = X is A, H, Q, R, K, D, N, Y, I, E, L, or T
  VARIANT 43: note = X is A, D, E, R, K, Q, H, N, or T
  VARIANT 44: note = X is an aromatic amino acid residue, a hydrophobic amino acid residue, or a neutral amino acid residue
  VARIANT 45: note = X is A, R, E, D, K, H, N, Q, or T
  VARIANT 46: note = X is A, H, E, R, D, K, I, L, N, Q, T, or Y
  VARIANT 47: note = X is L, A, D, K, I, N, or Q
  VARIANT 48: note = X is an aromatic amino acid residue, a hydrophobic amino acid residue, or a neutral amino acid residue
  VARIANT 49: note = X is E, D, K, H, N, Q, A, L, R, I, or Y
  VARIANT 50: note = X is A, N, Q, D, E, H, K, R, or S
  VARIANT 51: note = X is an aromatic amino acid residue, a hydrophobic amino acid residue, or a neutral amino acid residue
  VARIANT 52: note = X is S, A, P, or Q
  VARIANT 53: note = X is S, A, P, or Q
  VARIANT 54: note = X is A, L, I, K, D, N, Q, or R
MRGSHHHHHHGSIEGRVXXXXXXXXXXXXXXXXXXXXXXXXXXXXXXXXXXXXX

SEQ ID NO: 13 - protein, 54 aa, synthetic construct
  VARIANT 20: note = X is an aromatic amino acid residue, a hydrophobic amino acid residue, or a neutral amino acid residue
  VARIANT 23: note = X is an aromatic amino acid residue, a hydrophobic amino acid residue, or a neutral amino acid residue
  VARIANT 27: note = X is an aromatic amino acid residue, a hydrophobic amino acid residue, or a neutral amino acid residue
  VARIANT 30: note = X is an aromatic amino acid residue, a hydrophobic amino acid residue, or a neutral amino acid residue
  VARIANT 34: note = X is an aromatic amino acid residue, a hydrophobic amino acid residue, or a neutral amino acid residue
  VARIANT 37: note = X is an aromatic amino acid residue, a hydrophobic amino acid residue, or a neutral amino acid residue
  VARIANT 41: note = X is an aromatic amino acid residue, a hydrophobic amino acid residue, or a neutral amino acid residue
  VARIANT 44: note = X is an aromatic amino acid residue, a hydrophobic amino acid residue, or a neutral amino acid residue
  VARIANT 48: note = X is an aromatic amino acid residue, a hydrophobic amino acid residue, or a neutral amino acid residue
  VARIANT 51: note = X is an aromatic amino acid residue, a hydrophobic amino acid residue, or a neutral amino acid residue
MRGSHHHHHHGSIEGRVKEXTFXKNTXPQXLREXQEXNAAXQDXRELXRQXSKL

SEQ ID NO: 14 - protein, 54 aa, synthetic construct
  VARIANT 20: note = X is an aromatic amino acid residue, a hydrophobic amino acid residue, or a neutral amino acid residue
  VARIANT 23: note = X is an aromatic amino acid residue, a hydrophobic amino acid residue, or a neutral amino acid residue
  VARIANT 27: note = X is an aromatic amino acid residue, a hydrophobic amino acid residue, or a neutral amino acid residue
  VARIANT 30: note = X is an aromatic amino acid residue, a hydrophobic amino acid residue, or a neutral amino acid residue
  VARIANT 34: note = X is an aromatic amino acid residue, a hydrophobic amino acid residue, or a neutral amino acid residue
  VARIANT 37: note = X is an aromatic amino acid residue, a hydrophobic amino acid residue, or a neutral amino acid residue
  VARIANT 41: note = X is an aromatic amino acid residue, a hydrophobic amino acid residue, or a neutral amino acid residue
  VARIANT 48: note = X is an aromatic amino acid residue, a hydrophobic amino acid residue, or a neutral amino acid residue
  VARIANT 51: note = X is an aromatic amino acid residue, a hydrophobic amino acid residue, or a neutral amino acid residue
  VARIANT 44: note = X is an aromatic amino acid residue, a hydrophobic amino acid residue, or a neutral amino acid residue
MRGSHHHHHHGSIEGRVKEXLFXKKTXEQXLEEXKEXNKAXHDXRHLXENXSKL

SEQ ID NO: 15 - protein, 54 aa, synthetic construct
  VARIANT 20: note = X is an aromatic amino acid residue, a hydrophobic amino acid residue, or a neutral amino acid residue
  VARIANT 23: note = X is an aromatic amino acid residue, a hydrophobic amino acid residue, or a neutral amino acid residue
  VARIANT 27: note = X is an aromatic amino acid residue, a hydrophobic amino acid residue, or a neutral amino acid residue
  VARIANT 30: note = X is an aromatic amino acid residue, a hydrophobic amino acid residue, or a neutral amino acid residue
  VARIANT 34: note = X is an aromatic amino acid residue, a hydrophobic amino acid residue, or a neutral amino acid residue
  VARIANT 37: note = X is an aromatic amino acid residue, a hydrophobic amino acid residue, or a neutral amino acid residue
  VARIANT 41: note = X is an aromatic amino acid residue, a hydrophobic amino acid residue, or a neutral amino acid residue
  VARIANT 44: note = X is an aromatic amino acid residue, a hydrophobic amino acid residue, or a neutral amino acid residue
  VARIANT 48: note = X is an aromatic amino acid residue, a hydrophobic amino acid residue, or a neutral amino acid residue
  VARIANT 51: note = X is an aromatic amino acid residue, a hydrophobic amino acid residue, or a neutral amino acid residue
MRGSHHHHHHGSIEGRVKEXLFXKNTXYQXLLEXKEXNEAXYDXRHLXQQXSKL

SEQ ID NO: 16 - protein, 54 aa, synthetic construct
  VARIANT 20: note = X is an aromatic amino acid residue, a hydrophobic amino acid residue, or a neutral amino acid residue
  VARIANT 23: note = X is an aromatic amino acid residue, a hydrophobic amino acid residue, or a neutral amino acid residue
  VARIANT 27: note = X is an aromatic amino acid residue, a hydrophobic amino acid residue, or a neutral amino acid residue
  VARIANT 30: note = X is an aromatic amino acid residue, a hydrophobic amino acid residue, or a neutral amino acid residue
  VARIANT 34: note = X is an aromatic amino acid residue, a hydrophobic amino acid residue, or a neutral amino acid residue
  VARIANT 37: note = X is an aromatic amino acid residue, a hydrophobic amino acid residue, or a neutral amino acid residue
  VARIANT 41: note = X is an aromatic amino acid residue, a hydrophobic amino acid residue, or a neutral amino acid residue
  VARIANT 44: note = X is an aromatic amino acid residue, a hydrophobic amino acid residue, or a neutral amino acid residue
  VARIANT 48: note = X is an aromatic amino acid residue, a hydrophobic amino acid residue, or a neutral amino acid residue
  VARIANT 51: note = X is an aromatic amino acid residue, a hydrophobic amino acid residue, or a neutral amino acid residue
MRGSHHHHHHGSIEGRVKEXTFXKKTXEQXAEEXKEXNKAXHDXRHAXENXSKK

SEQ ID NO: 17 - protein, 54 aa, synthetic construct
  VARIANT 20: note = X is an aromatic amino acid residue, a hydrophobic amino acid residue, or a neutral amino acid residue
  VARIANT 23: note = X is an aromatic amino acid residue, a hydrophobic amino acid residue, or a neutral amino acid residue
  VARIANT 27: note = X is an aromatic amino acid residue, a hydrophobic amino acid residue, or a neutral amino acid residue
  VARIANT 30: note = X is an aromatic amino acid residue, a hydrophobic amino acid residue, or a neutral amino acid residue
  VARIANT 34: note = X is an aromatic amino acid residue, a hydrophobic amino acid residue, or a neutral amino acid residue
  VARIANT 37: note = X is an aromatic amino acid residue, a hydrophobic amino acid residue, or a neutral amino acid residue
  VARIANT 41: note = X is an aromatic amino acid residue, a hydrophobic amino acid residue, or a neutral amino acid residue
  VARIANT 44: note = X is an aromatic amino acid residue, a hydrophobic amino acid residue, or a neutral amino acid residue
  VARIANT 48: note = X is an aromatic amino acid residue, a hydrophobic amino acid residue, or a neutral amino acid residue
  VARIANT 51: note = X is an aromatic amino acid residue, a hydrophobic amino acid residue, or a neutral amino acid residue
MRGSHHHHHHGSIEGRVKEXTFXKNTXEQXAEEXKEXNKAXHDXRHQXENXSKQ

SEQ ID NO: 18 - protein, 54 aa, synthetic construct
  VARIANT 20: note = X is an aromatic amino acid residue, a hydrophobic amino acid residue, or a neutral amino acid residue
  VARIANT 23: note = X is an aromatic amino acid residue, a hydrophobic amino acid residue, or a neutral amino acid residue
  VARIANT 27: note = 30
  VARIANT 30: note = X is an aromatic amino acid residue, a hydrophobic amino acid residue, or a neutral amino acid residue
  VARIANT 34: note = X is an aromatic amino acid residue, a hydrophobic amino acid residue, or a neutral amino acid residue
  VARIANT 37: note = X is an aromatic amino acid residue, a hydrophobic amino acid residue, or a neutral amino acid residue
  VARIANT 41: note = X is an aromatic amino acid residue, a hydrophobic amino acid residue, or a neutral amino acid residue
  VARIANT 44: note = X is an aromatic amino acid residue, a hydrophobic amino acid residue, or a neutral amino acid residue
  VARIANT 48: note = X is an aromatic amino acid residue, a hydrophobic amino acid residue, or a neutral amino acid residue
  VARIANT 51: note = X is an aromatic amino acid residue, a hydrophobic amino acid residue, or a neutral amino acid residue
MRGSHHHHHHGSIEGRVKEXTFXKKQXEQXAEEXKEXNKAXHDXRHAXENXSKK

SEQ ID NO: 19 - protein, 54 aa, synthetic construct; identical to SEQ ID NO: 16
  VARIANT 20: note = X is an aromatic amino acid residue, a hydrophobic amino acid residue, or a neutral amino acid residue
  VARIANT 23: note = X is an aromatic amino acid residue, a hydrophobic amino acid residue, or a neutral amino acid residue
  VARIANT 27: note = X is an aromatic amino acid residue, a hydrophobic amino acid residue, or a neutral amino acid residue
  VARIANT 30: note = X is an aromatic amino acid residue, a hydrophobic amino acid residue, or a neutral amino acid residue
  VARIANT 34: note = X is an aromatic amino acid residue, a hydrophobic amino acid residue, or a neutral amino acid residue
  VARIANT 37: note = X is an aromatic amino acid residue, a hydrophobic amino acid residue, or a neutral amino acid residue
  VARIANT 41: note = X is an aromatic amino acid residue, a hydrophobic amino acid residue, or a neutral amino acid residue
  VARIANT 44: note = X is an aromatic amino acid residue, a hydrophobic amino acid residue, or a neutral amino acid residue
  VARIANT 48: note = X is an aromatic amino acid residue, a hydrophobic amino acid residue, or a neutral amino acid residue
  VARIANT 51: note = X is an aromatic amino acid residue, a hydrophobic amino acid residue, or a neutral amino acid residue

SEQ ID NO: 20 - protein, 54 aa, synthetic construct
  VARIANT 20: note = X is an aromatic amino acid residue, a hydrophobic amino acid residue, or a neutral amino acid residue
  VARIANT 23: note = X is an aromatic amino acid residue, a hydrophobic amino acid residue, or a neutral amino acid residue
  VARIANT 27: note = X is an aromatic amino acid residue, a hydrophobic amino acid residue, or a neutral amino acid residue
  VARIANT 30: note = X is an aromatic amino acid residue, a hydrophobic amino acid residue, or a neutral amino acid residue
  VARIANT 34: note = X is an aromatic amino acid residue, a hydrophobic amino acid residue, or a neutral amino acid residue
  VARIANT 37: note = X is an aromatic amino acid residue, a hydrophobic amino acid residue, or a neutral amino acid residue
  VARIANT 41: note = X is an aromatic amino acid residue, a hydrophobic amino acid residue, or a neutral amino acid residue
  VARIANT 44: note = X is an aromatic amino acid residue, a hydrophobic amino acid residue, or a neutral amino acid residue
  VARIANT 48: note = X is an aromatic amino acid residue, a hydrophobic amino acid residue, or a neutral amino acid residue
  VARIANT 51: note = X is an aromatic amino acid residue, a hydrophobic amino acid residue, or a neutral amino acid residue
MRGSHHHHHHGSIEGRVSEXTEXKKTXEHXAKEXKEXNKAXHTXRHAXENXAKN

SEQ ID NO: 21 - protein, 54 aa, synthetic construct
  VARIANT 20: note = X is an aromatic amino acid residue, a hydrophobic amino acid residue, or a neutral amino acid residue
  VARIANT 23: note = X is an aromatic amino acid residue, a hydrophobic amino acid residue, or a neutral amino acid residue
  VARIANT 27: note = X is an aromatic amino acid residue, a hydrophobic amino acid residue, or a neutral amino acid residue
  VARIANT 30: note = X is an aromatic amino acid residue, a hydrophobic amino acid residue, or a neutral amino acid residue
  VARIANT 34: note = X is an aromatic amino acid residue, a hydrophobic amino acid residue, or a neutral amino acid residue
  VARIANT 37: note = X is an aromatic amino acid residue, a hydrophobic amino acid residue, or a neutral amino acid residue
  VARIANT 41: note = X is an aromatic amino acid residue, a hydrophobic amino acid residue, or a neutral amino acid residue
  VARIANT 44: note = X is an aromatic amino acid residue, a hydrophobic amino acid residue, or a neutral amino acid residue
  VARIANT 48: note = X is an aromatic amino acid residue, a hydrophobic amino acid residue, or a neutral amino acid residue
  VARIANT 51: note = X is an aromatic amino acid residue, a hydrophobic amino acid residue, or a neutral amino acid residue
MRGSHHHHHHGSIEGRVKEXKFXKNTXPQXLREXQNXNMAXQDXRELXQQXSTL

SEQ ID NO: 22 - protein, 32 aa, synthetic construct
  VARIANT 1: note = X is an aromatic amino acid residue, a hydrophobic amino acid residue, or a neutral amino acid residue
  VARIANT 4: note = X is an aromatic amino acid residue, a hydrophobic amino acid residue, or a neutral amino acid residue
  VARIANT 8: note = X is an aromatic amino acid residue, a hydrophobic amino acid residue, or a neutral amino acid residue
  VARIANT 11: note = X is an aromatic amino acid residue, a hydrophobic amino acid residue, or a neutral amino acid residue
  VARIANT 15: note = X is an aromatic amino acid residue, a hydrophobic amino acid residue, or a neutral amino acid residue
  VARIANT 18: note = X is an aromatic amino acid residue, a hydrophobic amino acid residue, or a neutral amino acid residue
  VARIANT 22: note = X is an aromatic amino acid residue, a hydrophobic amino acid residue, or a neutral amino acid residue
  VARIANT 25: note = X is an aromatic amino acid residue, a hydrophobic amino acid residue, or a neutral amino acid residue
  VARIANT 29: note = X is an aromatic amino acid residue, a hydrophobic amino acid residue, or a neutral amino acid residue
  VARIANT 32: note = X is an aromatic amino acid residue, a hydrophobic amino acid residue, or a neutral amino acid residue
XTQXKNTXPQXLREXQNXNAAXQDXRELXQQX

SEQ ID NO: 23 - protein, 18 aa, synthetic construct
MRGSHHHHHHSIEGRVKE

SEQ ID NO: 24 - protein, 50 aa, synthetic construct
  VARIANT 19: note = X is an aromatic amino acid residue, a hydrophobic amino acid residue, or a neutral amino acid residue
  VARIANT 22: note = X is an aromatic amino acid residue, a hydrophobic amino acid residue, or a neutral amino acid residue
  VARIANT 26: note = X is an aromatic amino acid residue, a hydrophobic amino acid residue, or a neutral amino acid residue
  VARIANT 29: note = X is an aromatic amino acid residue, a hydrophobic amino acid residue, or a neutral amino acid residue
  VARIANT 33: note = X is an aromatic amino acid residue, a hydrophobic amino acid residue, or a neutral amino acid residue
  VARIANT 36: note = X is an aromatic amino acid residue, a hydrophobic amino acid residue, or a neutral amino acid residue
  VARIANT 40: note = X is an aromatic amino acid residue, a hydrophobic amino acid residue, or a neutral amino acid residue
  VARIANT 43: note = X is an aromatic amino acid residue, a hydrophobic amino acid residue, or a neutral amino acid residue
  VARIANT 47: note = X is an aromatic amino acid residue, a hydrophobic amino acid residue, or a neutral amino acid residue
  VARIANT 50: note = X is an aromatic amino acid residue, a hydrophobic amino acid residue, or a neutral amino acid residue
MRGSHHHHHHSIEGRVKEXTQXKNTXPQXLREXQNXNAAXQDXRELXQQX

SEQ ID NO: 25 - protein, 49 aa, synthetic construct
  VARIANT 4: note = X is an aromatic amino acid residue, a hydrophobic amino acid residue, or a neutral amino acid residue
  VARIANT 5: note = X is P or K
  VARIANT 6: note = X is Q or H
  VARIANT 7: note = X is an aromatic amino acid residue, a hydrophobic amino acid residue, or a neutral amino acid residue
  VARIANT 9: note = X is R or K
  VARIANT 11: note = X is an aromatic amino acid residue, a hydrophobic amino acid residue, or a neutral amino acid residue
  VARIANT 13: note = X is E or K
  VARIANT 14: note = X is an aromatic amino acid residue, a hydrophobic amino acid residue, or a neutral amino acid residue
  VARIANT 18: note = X is an aromatic amino acid residue, a hydrophobic amino acid residue, or a neutral amino acid residue
  VARIANT 20: note = X is D or S
  VARIANT 21: note = X is an aromatic amino acid residue, a hydrophobic amino acid residue, or a neutral amino acid residue
  VARIANT 23: note = X is E or T
  VARIANT 25: note = X is an aromatic amino acid residue, a hydrophobic amino acid residue, or a neutral amino acid residue
  VARIANT 26: note = X is R or Q
  VARIANT 28: note = X is an aromatic amino acid residue, a hydrophobic amino acid residue, or a neutral amino acid residue
  VARIANT 30: note = X is V or E
  VARIANT 32: note = X is an aromatic amino acid residue, a hydrophobic amino acid residue, or a neutral amino acid residue
  VARIANT 34: note = X is F or Q
  VARIANT 35: note = X is an aromatic amino acid residue, a hydrophobic amino acid residue, or a neutral amino acid residue
  VARIANT 37: note = X is D or E
  VARIANT 39: note = X is an aromatic amino acid residue, a hydrophobic amino acid residue, or a neutral amino acid residue
  VARIANT 40: note = X is M or E
  VARIANT 41: note = X is E or N
  VARIANT 42: note = X is an aromatic amino acid residue, a hydrophobic amino acid residue, or a neutral amino acid residue
  VARIANT 44: note = X is A or S
  VARIANT 46: note = X is an aromatic amino acid residue, a hydrophobic amino acid residue, or a neutral amino acid residue
  VARIANT 47: note = X is K or absent
  VARIANT 48: note = X is L or absent
  VARIANT 49: note = X is N or absent
MQLXXXXLXEXQXXNAAXQXXRXLXXQXVXEXTXXKXTXXXXDXSXXXX

(19) United States
(12) Patent Application Publication
(10) Pub. No.: US 2026/0116926 A1
(43) Pub. Date: Apr. 30, 2026

(54) PROTEIN-ENGINEERED PHOTORESPONSIVE CONDUCTIVE NANOFIBERS

(71) Applicant: New York University, New York, NY (US)

(72) Inventors: Jin Kim Montclare, New York, NY (US); Dustin Britton, Brooklyn, NY (US); Stephanie S. Lee, Hoboken, NJ (US)

(21) Appl. No.: 18/920,814

(22) Filed: Oct. 18, 2024

Related U.S. Application Data
(60) Provisional application No. 63/591,442, filed on Oct. 18, 2023.

(51) Int. Cl. C07K 14/00

(52) U.S. Cl. CPC C07K 14/001

(57) ABSTRACT

Provided are proteins and peptides having photoconductive characteristics. The proteins and peptides may have desirable self-assembling properties such that they form supramolecular structures (e.g., fibers or fibrils). The supramolecular structures may further gelate water such that a hydrogel is formed. The proteins and peptides may be used in devices having photoconductive characteristics. Such as, for example, memristors.

### CROSS REFERENCE TO RELATED APPLICATIONS

This application claims the bene fit of priority to U.S. Provisional Application No. 63/591,442, filed on Oct. 18, 2023, the disclosure of which is hereby incorporated by reference.

### STATEMENT REGARDING FEDERALLY SPONSORED RESEARCH

This invention was made with government support under 1728858 and 1420073 awarded by the National Science Foundation and W911NF-23-1-0269 awarded by the Army Research Office. The government has certain rights in the invention.

### SEQUENCE LISTING

The instant application contains a Sequence Listing, which has been submitted in .xml format and is hereby incorporated by reference in its entirety. Said .xml copy was created on Oct. 18, 2024, is named “058636_00756_ST26.xml”, and is 142,362 bytes in size.

### BACKGROUND OF THE DISCLOSURE

Photoconductive materials that convert photons into electrical current have important applications in photovoltaics, imaging, and biomedicine. Ultraviolet and infrared photodetectors typically use crystalline-Si and group III-V semiconductors that require expensive fabrication methods, such as atomic layer deposition and photolithography. Other materials include semiconducting polymers and poly-carbazoles that rely on uniform molecular organization to provide electron transport and conductivity. The improved performance of uniformly oriented metal nanoparticles, nanowires, and nanorods also highlight the importance of molecular organization in conductors and semiconductors in extant devices.

Nature has provided several examples of biological conductive materials that rely on ordered molecular assembly and organization. DNA is conductive and has even been modified with photo-triggerable motifs to create hybrid photoresponsive nanomaterials. Naturally occurring conductive proteins have remained tied to Geobacter sulfurreducens type IV pili. Its conductivity has been linked to secretion of polymerized c-type cytochrome filaments and tetrahaem packing cytochrome OmcE filaments. Previously, the electrically conductive properties of G. sulfurreducens had been attributed to the pi-pi stacking of its aromatic residues, which has inspired the development of engineered proteins and microbes for conduction. Other filamentous peptides using coiled-coils have demonstrated relatively high electrical conduction utilizing aromatic residues in the core to deliver electron transport via pi-pi stacking synthesized by fluorenylmethyloxycarbonyl (FMOC) chemistry. Engineering these bioelectronic materials can enable applications in fuel cells, biocompatible and portable power sources, miniature sensors, and neural interfaces, as have other semiconducting materials previously.

Imbuing self-assembling bioelectronic materials with photoresponsiveness will expand potential applications to data storage, optogenetics, and light-triggerable drug delivery. Many photoresponsive proteins have been engineered with photoswitchable chemicals. Self-assembling one-dimensional (1D) nanomaterials include pi-conjugated oligomers and polymers such as poly(3-alkylthiophene)s and poly(3,3″-didodecylquarterthiophene), and biomolecules such as DNA and proteins. Biomolecules, however, have only been investigated for their conducting and semiconducting properties as they are not inherently photoresponsive, whereas there exists many photoconductive conjugated polymers.

### SUMMARY OF THE DISCLOSURE

In an aspect, the present disclosure provides proteins or peptides. The proteins or peptides comprise a sequence designed by the methods described herein. The proteins and peptides may have photoconductive characteristics. The proteins and peptides may have desirable self-assembling properties such that they form supramolecular structures (e.g., fibers or fibrils). The supramolecular structures may further gelate water such that a hydrogel is formed.

For example, the proteins or peptides may be derived from a variant of a coiled-coil domain of cartilage oligomeric matrix protein (COMPcc), where the protein or peptide is photoconductive. The various proteins may be referred to as a “Photo-Q” protein/peptide followed by a number, where each number corresponds to a different protein/peptide. For example, the present disclosure provides Photo-Q1, Photo-Q2, Photo-Q3, Photo-Q4, Photo-Q5, Photo-Q6, Photo-Q7, Photo-Q8, Photo-Q9 or a protein comprising the sequences of Photo-Q1, Photo-Q2, Photo-Q3, Photo-Q4, Photo-Q5, Photo-Q6, Photo-Q7, Photo-Q8, or Photo-Q9. For simplicity, as used herein, these peptides may simply be referred to as Q1, Q2, Q3, Q4, Q5, Q6, Q7, Q8, or Q9. Also provided are peptides and proteins that may be referred to as CHAF and L-CHAF. Also provided is a photoconductive analog of COMPcc, which may be referred to as “photo-C.” However, as used herein, photo-C may be simply referred to as “C.”

Photo-Q proteins or peptides may have or comprise the following sequence:

(SEQ ID NO: 1)
VX₁X₂X₃X₄X₅X₆X₇X₈X₉X₁₀X₁₁X₁₂X₁₃X₁₄X₁₅X₁₆X₁₇X₁₈
X₁₉X₂₀X₂₁X₂₂X₂₃X₂₄X₂₅X₂₆X₂₇X₂₈X₂₉X₃₀X₃₁X₃₂X₃₃
X₃₄X₃₅X₃₆X₃₇,

where X₃, X₆, X₁₀, X₁₃, X₁₇, X₂₀, X₂₄, X₂₇, X₃₁, and X₃₄ are each independently an aromatic amino acid residue, a hydrophobic amino acid residue, or a neutral amino acid residue;
X₁ is A, E, D, R, H, K, Q, N, or S;
X₂ is A, E, N, or Q;
X₄ is A, E, R, D, H, I, L, T, K, Q, or N;
X₅ is A, F, Q, R, K, H, D, S, or E;
X₇ is A, K, or E;
X₈ is A, K, E, D, R, H, Q, or N;
X₉ is A, T, I, L, or Q;
X₁₁ is A, E, D, H, P, I, L, K, Y, N, Q, or R;
X₁₂ is A, Q, H, E, D, K, R, or N;
X₁₄ is A, L, D, E, K, or I;
X₁₅ is A, E, D, H, Y, I, L, R, K, Q, or N;
X₁₆ is A, E, or Q;
X₁₈ is A, K, E, D, K, R, H, N, or Q;
X₁₉ is A, N, D, K, R, H, Q, or E;
X₂₁ is A, N, or Q;
X₂₂ is K, A, E, I, L, M, R, H, D, Q, N, or S;
X₂₃ is A, Q, N, I, or L;
X₂₅ is A, H, Q, R, K, D, N, Y, I, E, L, or T;
X₂₆ is A, D, E, R, K, Q, H, N, or T;
X₂₈ is A, R, E, D, K, H, N, Q, or T;
X₂₉ is A, H, E, R, D, K, I, L, N, Q, T, or Y;
X₃₀ is L, A, D, K, I, N, or Q;
X₃₂ is E, D, K, H, N, Q, A, L, R, I, or Y;
X₃₃ is A, N, Q, D, E, H, K, R, or S;
X₃₅ is S, A, P, or Q;
X₃₆ is A, K, T, D, R, H, N, Q, or E; and
X₃₇ is A, L, I, K, D, N, Q, or R.
In various examples, at least one of X₃, X₆, X₁₀, X₁₃, X₁₇, X₂₀, X₂₄, X₂₇, X₃₁, and X₃₄ is an aromatic residue

In various examples, a CHAF protein or peptide may have or comprise the following sequence:

(SEQ ID NO: 22)
X₃₈TQX₃₉KNTX₄₀PQX₄₁LREX₄₂QNX₄₃NAAX₄₄QD
X₄₅RELX₄₆QQX₄₇,

where X₃₈, X₃₉, X₄₀, X₄₁, X₄₂, X₄₃, X₄₄, X₄₅, X₄₆, and X₄₇ are each independently an aromatic amino acid residue, a hydrophobic amino acid residue, or a neutral amino acid residue. In various examples, at least one of X₃₈, X₃₉, X₄₀, X₄₁, X₄₂, X₄₃, X₄₄, X₄₅, X₄₆, and X₄₇ is an aromatic residue.

In various examples, a protein or peptide may have or comprise the following sequence:

(SEQ ID NO: 25)
MQLX₄₈X₄₉X₅₀X₅₁LX₅₂EX₅₃QX₅₄X₅₅NAAX₅₆QX₅₇X₅₈RX₅₉
LX₆₀X₆₁QX₆₂VX₆₃EX₆₄TX₆₅X₆₆KX₆₇TX₆₈X₆₉X₇₀X₇₁DX₇₂
SX₇₃X₇₄X₇₅X₇₆,

where X₄₈, X₅₁, X₅₃, X₅₅, X₅₆, X₅₈, X₆₀, X₆₂, X₆₄, X₆₆, X₆₈, X₇₁, and X₇₃ are each independently an aromatic amino acid residue, a hydrophobic amino acid residue, or a neutral amino acid residue;
X₄₉ is P or K;
X₅₀ is Q or H;
X₅₂ is R or K;
X₅₄ is E or K;
X₅₇ is D or S;
X₅₉ is E or T;
X₆₁ is R or Q;
X₆₃ is V or E;
X₆₅ is F or Q;
X₆₇ is D or E;
X₆₉ is M or E;
X₇₀ is E or N;
X₇₂ is A or S;
X₇₄ is K or absent;
X₇₅ is L or absent; and
X₇₆ is N or absent.
In various examples, at least one of X₄₈, X₅₁, X₅₃, X₅₅, X₅₆, X₅₈, X₆₀, X₆₂, X₆₄, X₆₆, X₆₈, X₇₁, and X₇₃ is an aromatic residue.

In an aspect, the present disclosure provides protein fibers or peptide fibers and gels comprising those fibers. The protein fibers and peptide fibers may be referred to fibers. These fibers may comprise one or more protofibers. The one or more protofibers may comprise a plurality of proteins and/or peptides of the present disclosure.

In an aspect, the present disclosure provides devices. A device may comprise a protein or peptide of the present disclosure or protein or peptide fibers of the present disclosure.

### BRIEF DESCRIPTION OF THE FIGURES

For a fuller understanding of the nature and objects of the disclosure, reference should be made to the following detailed description taken in conjunction with the accompanying figures.

FIG. 1. CHAF/L-CHAF protein sequence (SEQ ID NO:13) labelled by helical wheel positions a-g. CHAF protein possesses F in the a and d positions whereas L-CHAF protein possess L in the a and d positions. Cartoon of CHAF protein is displayed as a ribbon diagram with phenylalanines highlighted as sticks and as an electrostatic potential map from N- to C-terminus. Positive blue and negative red patches represent a scale of −10 to 10 kbT. Schematic represents the proposed self-assembly by predominant end-to-end stacking of coiled-coils.

FIG. 2. Representative electron microscopy images of CHAF protein fibers at increasing resolution using a-b) SEM and c-d) TEM. Average CD wavelength scan of e) CHAF and f) L-CHAF from three independent trials. g) Average secondary structure content of CHAF, L-CHAF, denatured CHAF, and denatured L-CHAF by ATR-FTIR deconvolution from three independent trials. ATR-FTIR secondary structure content is color-coded with α-helical structure in red (left in each series), β-sheet structure in blue (middle in each series), and random coil structure in green (right in each series). * represents p-value <0.05, ** represents p-value <0.01, *** represents p-value <0.001.

FIG. 3. a) Scheme of photodetector setup. Inset: POM of a dropcast CHAF film (scale bar=100 μm). b) CHAF photodetector response to 808 nm light at intensities ranging from 0-1.28 mW/cm² at applied biases ranging from −10 to 10 V.

FIG. 4. Time-dependent photocurrent during 25 s cycles of the excitation light being turned on and off at intensities ranging from 0 to 1.28 mW/cm² for a) CHAF photodetectors, b) L-CHAF photodetectors, c) annealed CHAF photodetectors and d) annealed L-CHAF photodetectors.

FIG. 5. Leading natural transition orbitals (NTO) for electronic excitation from the ground state (S₀) with their corresponding weights (Wt.). An isovalue of 0.02 was used to plot each surface. The energy of the excited state was calculated with respect to the ground state (S₀). The systems were rotated for better visualization of each NTO.

FIG. 6. Proposed mechanism of initial excited states (Sᵢ) formation with phenylalanine (PHE) as the redox cofactor.

FIG. 7. Schematic showing irradiation of a protein of the present disclosure.

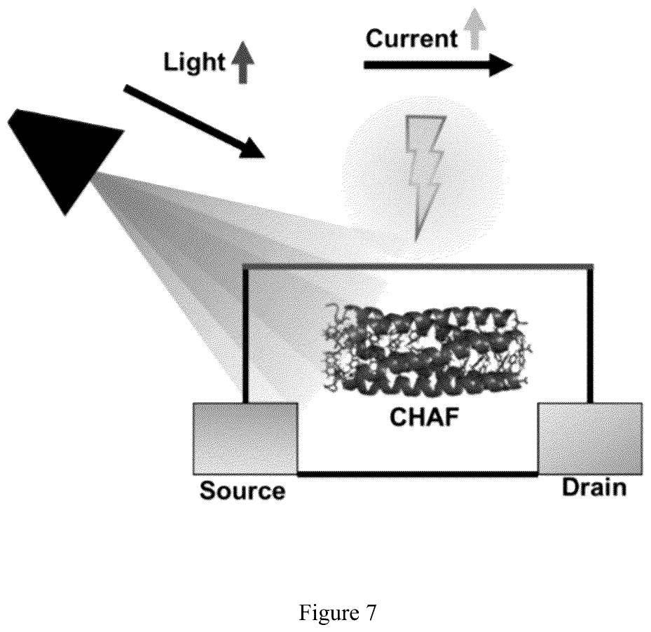

FIG. 8. a Example of Monte-Carlo search to remove electrostatic potential from the N- and C-terminus. Results show calculation of the N- and C-terminus E_{bcf} and Rosetta score after iterative mutations using Rosetta to randomly mutate b, c, and f helical wheel positions. b Example of Monte-Carlo search results to add electrostatic potential to the very ends of the N- and C-termini. E_{bcf} is calculated for only the first three residues from the N- or C-terminus of the coiled-coil.

FIG. 9. CHAF protein (6.29 kDa) after purification. L: Ladder, FT: Flow-through, following are increasing concentrations of imidazole.

FIG. 10. L-CHAF protein (5.90 kDa) after purification. L: Ladder, FT: Flow-through, following are increasing concentrations of imidazole.

FIG. 11. Representative TEM images of L-CHAF at various resolutions.

FIG. 12. a. Representative ATR-FTIR spectra of the amide I bond region for CHAF. b. Representative ATR-FTIR spectra of the amide I bond region for L-CHAF. c. Representative ATR-FTIR spectra of the amide I bond region for denatured CHAF. d. Representative ATR-FTIR spectra of the amide I bond region for denatured L-CHAF. Deconvoluted peaks are colored: red for α-helix, blue for β-sheet, and green for random coil.

FIG. 13. Representative SEM image of 40 μM protein deposited at the Au electrode (top)/glass substrate (bottom) interface of a photodetector device.

FIG. 14. Representative POM images of the two-terminal devices fabricated using the dropcast CHAF film. The scale bars in a,b) are 400 and 40 μm, respectively.

FIG. 15. CHAF photodetector response to 365 nm light at intensities ranging from 0-1.3 mW/cm² at applied biases ranging from −3 to 3 V.

FIG. 16. Representative POM image of CHAF film after thermal annealing.

FIG. 17. Residues for redox potential calculations. (a) Position of the selected Phe and His residues in the assembly. QM/MM setup for single-point calculations with sidechains of (b) Phe and (c) His. The QM region is highlighted.

FIG. 18. Sample preparation to determine the IR response of single protein fibers.

FIG. 19. XRD spectra for fibers prepared via spin coating and for fibers prepared via drop-casting.

FIG. 20. A plot showing intensity versus q.

FIG. 21. Device fabrication for a protein single fiber photodetector.

FIG. 22. Results from photodetector performance.

FIG. 23. Memristor performance of protein fibers of the present disclosure.

FIG. 24. Sequence of voltage sweep.

FIG. 25. Sequence of voltage sweep and memristor characteristics of protein fibers of the present disclosure.

FIG. 26. Data from current measurements.

FIG. 27. Data from current measurements.

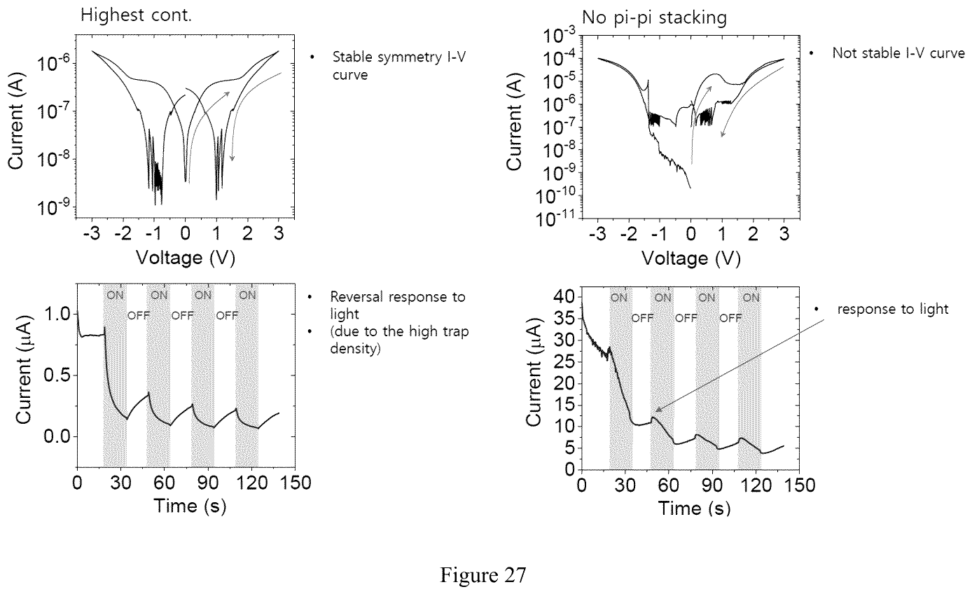

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g., 90%, 95%, or more confidence interval from the mean), such as, for example, variations of +/−10% or less, +/−5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%, 0.5% to 2.4%, 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about”, it will be understood that the particular value forms a further disclosure. For example, if the value “about 10” is disclosed, then “10” is also disclosed.

As used herein, the terms “including,” “containing,” and “comprising” are used in their open, non-limiting sense.

As used in this disclosure, the singular forms include the plural forms and vice versa unless the context clearly indicates otherwise.

The articles “a” and “an” are used in this disclosure to refer to one or more than one (i.e., to at least one) of the grammatical object of the article. By way of example, “an element” means one element or more than one element.

As used herein, unless otherwise stated or indicated, “s” refers to second(s), “min” refers to minute(s), and “h” refers to hour(s).

The phrase “therapeutically effective amount” is used herein to mean an amount sufficient to reduce by at least about 15 percent, preferably by at least 50 percent, more preferably by at least 90 percent, and most preferably prevents oxidative stress in the individual. Alternatively, a therapeutically effective amount is sufficient to cause an improvement in a clinically significant condition in the individual.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent, trivalent, and the like, radicals). Illustrative examples of groups include:

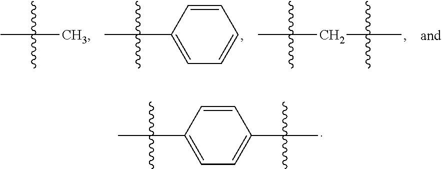

Amino acids and amino acid residues may be referred to herein by either their commonly known three letter symbols or by the one-letter symbols recommended by the IUPAC-IUB Biochemical Nomenclature Commission.

The present disclosure also provides sequences that have identity with the protein or peptides sequences (including antibody sequences) described herein. In various examples, the sequences have at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity with a protein or peptide sequence of the present disclosure.

In an aspect, the present disclosure provides proteins or peptides. The proteins or peptides comprise a sequence designed by the methods described herein. The proteins and peptides may have photoconductive characteristics. The proteins and peptides may have desirable self-assembling properties such that they form supramolecular structures (e.g., fibers or fibrils). The supramolecular structures may further gelate water such that a hydrogel is formed.

For example, the proteins or peptides may be derived from a variant of a coiled-coil domain of cartilage oligomeric matrix protein (COMPcc), where the protein or peptide is photoconductive. COMPcc and other peptides are disclosed in U.S. patent application Ser. No. 18/460,519 (U.S. Pat. Pub. No. 2024/0115742), the disclosure of which is incorporated herein in its entirety. The various proteins of the present disclosure may be referred to as a “Photo-Q” protein/peptide followed by a number, where each number corresponds to a different protein/peptide. For example, the present disclosure provides Photo-Q1, Photo-Q2, Photo-Q3, Photo-Q4, Photo-Q5, Photo-Q6, Photo-Q7, Photo-Q8, Photo-Q9 or a protein comprising the sequences of Photo-Q1, Photo-Q2, Photo-Q3, Photo-Q4, Photo-Q5, Photo-Q6, Photo-Q7, Photo-Q8, or Photo-Q9. For simplicity, as used herein, these peptides may simply be referred to as Q1, Q2, Q3, Q4, Q5, Q6, Q7, Q8, or Q9. Also provided are peptides and proteins that may be referred to as CHAF and L-CHAF. Also provided is a photoconductive analog of COMPcc, which may be referred to as “photo-C.” However, as used herein, photo-C may be simply referred to as “C.”

Photo-Q proteins or peptides may have or comprise the following sequence:

(SEQ ID NO: 1)
VX₁X₂X₃X₄X₅X₆X₇X₈X₉X₁₀X₁₁X₁₂X₁₃X₁₄X₁₅X₁₆X₁₇X₁₈
X₁₉X₂₀X₂₁X₂₂X₂₃X₂₄X₂₅X₂₆X₂₇X₂₈X₂₉X₃₀X₃₁X₃₂X₃₃X₃₄
X₃₅X₃₆X₃₇,

where X₃, X₆, X₁₀, X₁₃, X₁₇, X₂₀, X₂₄, X₂₇, X₃₁, and X₃₄ are each independently an aromatic amino acid residue, a hydrophobic amino acid residue, or a neutral amino acid residue;
X₁ is A, E, D, R, H, K, Q, N, or S;
X₂ is A, E, N, or Q;
X₄ is A, E, R, D, H, I, L, T, K, Q, or N;
X₅ is A, F, Q, R, K, H, D, S, or E;
X₇ is A, K, or E;
X₈ is A, K, E, D, R, H, Q, or N;
X₉ is A, T, I, L, or Q;
X₁₁ is A, E, D, H, P, I, L, K, Y, N, Q, or R;
X₁₂ is A, Q, H, E, D, K, R, or N;
X₁₄ is A, L, D, E, K, or I;
X₁₅ is A, E, D, H, Y, I, L, R, K, Q, or N;
X₁₆ is A, E, or Q;
X₁₈ is A, K, E, D, K, R, H, N, or Q;
X₁₉ is A, N, D, K, R, H, Q, or E;
X₂₁ is A, N, or Q;
X₂₂ is K, A, E, I, L, M, R, H, D, Q, N, or S;
X₂₃ is A, Q, N, I, or L;
X₂₅ is A, H, Q, R, K, D, N, Y, I, E, L, or T;
X₂₆ is A, D, E, R, K, Q, H, N, or T;
X₂₈ is A, R, E, D, K, H, N, Q, or T;
X₂₉ is A, H, E, R, D, K, I, L, N, Q, T, or Y;
X₃₀ is L, A, D, K, I, N, or Q;
X₃₂ is E, D, K, H, N, Q, A, L, R, I, or Y;
X₃₃ is A, N, Q, D, E, H, K, R, or S;
X₃₅ is S, A, P, or Q;
X₃₅ is S, A, P, or Q; and
X₃₇ is A, L, I, K, D, N, Q, or R.
In various examples, at least one of X₃, X₆, X₁₀, X₁₃, X₁₇, X₂₀, X₂₄, X₂₇, X₃₁, and X₃₄ is an aromatic residue.

Aromatic amino acid residues may be F, Y, or W. Hydrophobic amino acid residues may be L, I, V, M, or A. Neutral amino acid residues may be Q, N, T, or S. In various examples, at least one (e.g., one or more or all) of X₃, X₆, X₁₀, X₁₃, X₁₇, X₂₀, X₂₄, X₂₇, X₃₁, and X₃₄ is an aromatic residue (e.g., F) and the remaining aforementioned residues are hydrophobic residues or neutral residues. In various examples, at least one (e.g., one or more or all) of X₃, X₆, X₁₀, X₁₃, X₁₇, X₂₀, X₂₄, X₂₇, X₃₁, and X₃₄ is an aromatic residue (e.g., F) and the remaining aforementioned residues are hydrophobic residues. In various examples, X₃, X₆, X₁₀, X₁₃, X₁₇, X₂₀, X₂₄, X₂₇, X₃₁, and X₃₄ may each independently be F or L. In various examples, X₃, X₆, X₁₀, X₁₃, X₁₇, X₂₀, X₂₄, X₂₇, X₃₁, and X₃₄ are all L. In various examples, X₃, X₆, X₁₀, X₁₃, X₁₇, X₂₀, X₂₄, X₂₇, X₃₁, and X₃₄ are all F.

A protein or peptide of the present disclosure may have or comprise the following sequence:
VKEX₃TFX₆KNTX₁₀PQX₁₃LREX₁₇QEX₂₀NAAX₂₄QDX₂₇RELX₃₁RQX₃₄SKL (SEQ ID NO:2);
VKEX₃LFX₆KKTX₁₀EQX₁₃LEEX₁₇KEX₂₀NKAX₂₄HDX₂₇RHLX₃₁ENX₃₄SKL (SEQ ID NO:3);
VKEX₃LFX₆KNTX₁₀YQX₁₃LLEX₁₇KEX₂₀NEAX₂₄YDX₂₇RHLX₃₁QQX₃₄SKL (SEQ ID NO:4);
VKEX₃TFX₆KKTX₁₀EQX₁₃AEEX₁₇KEX₂₀NKAX₂₄HDX₂₇RHAX₃₁ENX₃₄SKK (SEQ ID NO:5);
VKEX₃TFX₆KNTX₁₀EQX₁₃AEEX₁₇KEX₂₀NKAX₂₄HDX₂₇RHQX₃₁ENX₃₄SKQ (SEQ ID NO:6);
VKEX₃TFX₆KKQX₁₀EQX₁₃AEEX₁₇KEX₂₀NKAX₂₄HDX₂₇RHAX₃₁ENX₃₄SKK (SEQ ID NO:7);
VKEX₃TFX₆KKTX₁₀EQX₁₃AEEX₁₇KEX₂₀NKAX₂₄HDX₂₇RHAX₃₁ENX₃₄SKK (SEQ ID NO:8);
VSEX₃TEX₆KKTX₁₀EHX₁₃AKEX₁₇KEX₂₀NKAX₂₄HTX₂₇RHAX₃₁ENX₃₄AKN (SEQ ID NO: 9); or
VKEX₃KFX₆KNTX₁₀PQX₁₃LREX₁₇QNX₂₀NMAX₂₄QDX₂₇RELX₃₁QQX₃₄STL (SEQ ID NO:10), where X₃, X₆, X₁₀, X₁₃, X₁₇, X₂₀, X₂₄, X₂₇, X₃₁, and X₃₄ are each independently an aromatic amino acid residue, a hydrophobic amino acid residue, or a neutral amino acid residue. In various examples, at least one (e.g., one or more or all) of X₃, X₆, X₁₀, X₁₃, X₁₇, X₂₀, X₂₄, X₂₇, X₃₁, and X₃₄ is an aromatic residue (e.g., F) and the remaining aforementioned residues are hydrophobic residues or neutral residues. In various examples, at least one (e.g., one or more or all) of X₃, X₆, X₁₀, X₁₃, X₁₇, X₂₀, X₂₄, X₂₇, X₃₁, and X₃₄ is an aromatic residue (e.g., F) and the remaining aforementioned residues are hydrophobic residues.

In various examples, the protein or peptide may further comprise the following sequence conjugated to the N-terminus: MRGSHHHHHHGSIEGR (SEQ ID NO:11). Thus, for example, the protein or peptide of the present disclosure may have the following sequence:

(SEQ ID NO: 12)
MRGSHHHHHHGSIEGRVX₁X₂X₃X₄X₅X₆X₇X₈X₉X₁₀
X₁₁X₁₂X₁₃X₁₄X₁₅X₁₆X₁₇X₁₈X₁₉X₂₀X₂₁X₂₂X₂₃X₂₄X₂₅
X₂₆X₂₇X₂₈X₂₉X₃₀X₃₁X₃₂X₃₃X₃₄X₃₅X₃₆X₃₇,

where X₃, X₆, X₁₀, X₁₃, X₁₇, X₂₀, X₂₄, X₂₇, X₃₁, and X₃₄ are each independently an aromatic amino acid residue, a hydrophobic amino acid residue, or a neutral amino acid residue;
X₁ is A, E, D, R, H, K, Q, N, or S;
X₂ is A, E, N, or Q;
X₄ is A, E, R, D, H, I, L, T, K, Q, or N;
X₅ is A, F, Q, R, K, H, D, S, or E;
X₇ is A, K, or E;
X₈ is A, K, E, D, R, H, Q, or N;
X₉ is A, T, I, L, or Q;
X₁₁ is A, E, D, H, P, I, L, K, Y, N, Q, or R;
X₁₂ is A, Q, H, E, D, K, R, or N;
X₁₄ is A, L, D, E, K, or I;
X₁₅ is A, E, D, H, Y, I, L, R, K, Q, or N;
X₁₆ is A, E, or Q;
X₁₈ is A, K, E, D, K, R, H, N, or Q;
X₁₉ is A, N, D, K, R, H, Q, or E;
X₂₁ is A, N, or Q;
X₂₂ is K, A, E, I, L, M, R, H, D, Q, N, or S;
X₂₃ is A, Q, N, I, or L;
X₂₅ is A, H, Q, R, K, D, N, Y, I, E, L, or T;
X₂₆ is A, D, E, R, K, Q, H, N, or T;
X₂₈ is A, R, E, D, K, H, N, Q, or T;
X₂₉ is A, H, E, R, D, K, I, L, N, Q, T, or Y;
X₃₀ is L, A, D, K, I, N, or Q;
X₃₂ is E, D, K, H, N, Q, A, L, R, I, or Y;
X₃₃ is A, N, Q, D, E, H, K, R, or S;
X₃₅ is S, A, P, or Q;
X₃₅ is S, A, P, or Q; and
X₃₇ is A, L, I, K, D, N, Q, or R.
In various examples, at least one (e.g., one or more or all) of X₃, X₆, X₁₀, X₁₃, X₁₇, X₂₀, X₂₄, X₂₇, X₃₁, and X₃₄ is an aromatic residue (e.g., F) and the remaining aforementioned residues are hydrophobic residues or neutral residues. In various examples, at least one (e.g., one or more or all) of X₃, X₆, X₁₀, X₁₃, X₁₇, X₂₀, X₂₄, X₂₇, X₃₁, and X₃₄ is an aromatic residue (e.g., F) and the remaining aforementioned residues are hydrophobic residues.

A protein or peptide of the present disclosure may have or comprise the following sequence:

(SEQ ID NO: 13)
MRGSHHHHHHGSIEGRVKEX₃TFX₆KNTX₁₀PQX₁₃LREX₁₇QE
X₂₀NAAX₂₄QDX₂₇RELX₃₁RQX₃₄SKL;
(SEQ ID NO: 14)
MRGSHHHHHHGSIEGRVKEX₃LFX₆KKTX₁₀EQX₁₃LEEX₁₇KE
X₂₀NKAX₂₄HDX₂₇RHLX₃₁ENX₃₄SKL;
(SEQ ID NO: 15)
MRGSHHHHHHGSIEGRVKEX₃LFX₆KNTX₁₀YQX₁₃LLEX₁₇KE
X₂₀NEAX₂₄YDX₂₇RHLX₃₁QQX₃₄SKL;
(SEQ ID NO: 16)
MRGSHHHHHHGSIEGRVKEX₃TFX₆KKTX₁₀EQX₁₃AEEX₁₇KE
X₂₀NKAX₂₄HDX₂₇RHAX₃₁ENX₃₄SKK;
(SEQ ID NO: 17)
MRGSHHHHHHGSIEGRVKEX₃TFX₆KNTX₁₀EQX₁₃AEEX₁₇KE
X₂₀NKAX₂₄HDX₂₇RHQX₃₁ENX₃₄SKQ;
(SEQ ID NO: 18)
MRGSHHHHHHGSIEGRVKEX₃TFX₆KKQX₁₀EQX₁₃AEEX₁₇KE
X₂₀NKAX₂₄HDX₂₇RHAX₃₁ENX₃₄SKK;
(SEQ ID NO: 19)
MRGSHHHHHHGSIEGRVKEX₃TFX₆KKTX₁₀EQX₁₃AEEX₁₇KE
X₂₀NKAX₂₄HDX₂₇RHAX₃₁ENX₃₄SKK;
(SEQ ID NO: 20)
MRGSHHHHHHGSIEGRVSEX₃TEX₆KKTX₁₀EHX₁₃AKEX₁₇KE
X₂₀NKAX₂₄HTX₂₇RHAX₃₁ENX₃₄AKN;
or
(SEQ ID NO: 21)
MRGSHHHHHHGSIEGRVKEX₃KFX₆KNTX₁₀PQX₁₃LREX₁₇QN
X₂₀NMAX₂₄QDX₂₇RELX₃₁QQX₃₄STL,

where X₃, X₆, X₁₀, X₁₃, X₁₇, X₂₀, X₂₄, X₂₇, X₃₁, and X₃₄ are each independently an aromatic amino acid residue, a hydrophobic amino acid residue, or a neutral amino acid residue. In various examples, at least one (e.g., one or more or all) of X₃, X₆, X₁₀, X₁₃, X₁₇, X₂₀, X₂₄, X₂₇, X₃₁, and X₃₄ is an aromatic residue (e.g., F) and the remaining aforementioned residues are hydrophobic residues or neutral residues. In various examples, at least one (e.g., one or more or all) of X₃, X₆, X₁₀, X₁₃, X₁₇, X₂₀, X₂₄, X₂₇, X₃₁, and X₃₄ is an aromatic residue (e.g., F) and the remaining aforementioned residues are hydrophobic residues.

In various examples, a CHAF protein or peptide may have or comprise the following sequence:

(SEQ ID NO: 22)
X₃₈TQX₃₉KNTX₄₀PQX₄₁LREX₄₂QNX₄₃NAAX₄₄QDX₄₅R
ELX₄₆QQX₄₇,

where X₃₈, X₃₉, X₄₀, X₄₁, X₄₂, X₄₃, X₄₄, X₄₅, X₄₆, and X₄₇ are each independently an aromatic amino acid residue, a hydrophobic amino acid residue, or a neutral amino acid residue. In various examples, at least one of X₃₈, X₃₉, X₄₀, X₄₁, X₄₂, X₄₃, X₄₄, X₄₅, X₄₆, and X₄₇ is an aromatic residue

Aromatic amino acid residues may be F, Y, or W. Hydrophobic amino acid residues may be L, I, V, M, or A. Neutral amino acid residues may be Q, N, T, or S. In various examples, at least one (e.g., one or more or all) of X₃₈, X₃₉, X₄₀, X₄₁, X₄₂, X₄₃, X₄₄, X₄₅, X₄₆, and X₄₇ is an aromatic residue (e.g., F) and the remaining aforementioned residues are hydrophobic residues or neutral residues. In various examples, at least one (e.g., one or more or all) of X₃₈, X₃₉, X₄₀, X₄₁, X₄₂, X₄₃, X₄₄, X₄₅, X₄₆, and X₄₇ is an aromatic residue (e.g., F) and the remaining aforementioned residues are hydrophobic residues. In various examples, X₃₈, X₃₉, X₄₀, X₄₁, X₄₂, X₄₃, X₄₄, X₄₅, X₄₆, and X₄₇ may each independently be F or L. In various examples, X₃₈, X₃₉, X₄₀, X₄₁, X₄₂, X₄₃, X₄₄, X₄₅, X₄₆, and X₄₇ are all L. In various examples, X₃₈, X₃₉, X₄₀, X₄₁, X₄₂, X₄₃, X₄₄, X₄₅, X₄₆, and X₄₇ are all F. Without intending to be bound by any particular theory, when all of X₃₈, X₃₉, X₄₀, X₄₁, X₄₂, X₄₃, X₄₄, X₄₅, X₄₆, and X₄₇ are aromatic (e.g., F), it is expected the resulting peptide has a more desirable photoconductivity than when all of X₃₈, X₃₉, X₄₀, X₄₁, X₄₂, X₄₃, X₄₄, X₄₅, X₄₆, and X₄₇ are L.

In various examples, a protein or peptide of the present disclosure may further comprise the following sequence conjugated to the N-terminus:
MRGSHHHHHHSIEGRVKE (SEQ ID NO:23). Thus, the resulting protein or peptide may have or comprise the following sequence:

(SEQ ID NO: 24)
MRGSHHHHHHSIEGRVKEX₃₈TQX₃₉KNTX₄₀PQX₄₁LRE
X₄₂QNX₄₃NAAX₄₄QDX₄₅RELX₄₆QQX₄₇.

In various examples, a protein or peptide may have or comprise the following sequence:

(SEQ ID NO: 25)
MQLX₄₈X₄₉X₅₀X₅₁LX₅₂EX₅₃QX₅₄X₅₅NAAX₅₆QX₅₇X₅₈RX₅₉LX₆₀X₆₁QX₆₂
VX₆₃EX₆₄TX₆₅X₆₆KX₆₇TX₆₈X₆₉X₇₀X₇₁DX₇₂SX₇₃X₇₄X₇₅X₇₆,

where X₄₈, X₅₁, X₅₃, X₅₅, X₅₆, X₅₈, X₆₀, X₆₂, X₆₄, X₆₆, X₆₈, X₇₁, and X₇₃ are each independently an aromatic amino acid residue, a hydrophobic amino acid residue, or a neutral amino acid residue;
X₄₉ is P or K;
X₅₀ is Q or H;
X₅₂ is R or K;
X₅₄ is E or K;
X₅₇ is D or S;
X₅₉ is E or T;
X₆₁ is R or Q;
X₆₃ is V or E;
X₆₅ is F or Q;
X₆₇ is D or E;
X₆₉ is M or E;
X₇₀ is E or N;
X₇₂ is A or S;
X₇₄ is K or absent;
X₇₅ is L or absent; and
X₇₆ is N or absent.

Aromatic amino acid residues may be F, Y, or W. Hydrophobic amino acid residues may be L, I, V, M, or A. Neutral amino acid residues may be Q, N, T, or S. In various examples, at least one (e.g., one or more or all) of X₄₈, X₅₁, X₅₃, X₅₅, X₅₆, X₅₈, X₆₀, X₆₂, X₆₄, X₆₆, X₆₈, X₇₁, and X₇₃ is an aromatic residue (e.g., F) and the remaining aforementioned residues are hydrophobic residues or neutral residues. In various examples, at least one (e.g., one or more or all) of X₄₈, X₅₁, X₅₃, X₅₅, X₅₆, X₅₈, X₆₀, X₆₂, X₆₄, X₆₆, X₆₈, X₇₁, and X₇₃ is an aromatic residue (e.g., F) and the remaining aforementioned residues are hydrophobic residues. In various examples, X₄₈, X₅₁, X₅₃, X₅₅, X₅₆, X₅₈, X₆₀, X₆₂, X₆₄, X₆₆, X₆₈, X₇₁, and X₇₃ may each independently be F or L. In various examples, X₄₈, X₅₁, X₅₃, X₅₅, X₅₆, X₅₈, X₆₀, X₆₂, X₆₄, X₆₆, X₆₈, X₇₁, and X₇₃ are all L. In various examples, X₄₈, X₅₁, X₅₃, X₅₅, X₅₆, X₅₈, X₆₀, X₆₂, X₆₄, X₆₆, X₆₈, X₇₁, and X₇₃ are all F. Without intending to be bound by any particular theory, when all of X₄₈, X₅₁, X₅₃, X₅₅, X₅₆, X₅₈, X₆₀, X₆₂, X₆₄, X₆₆, X₆₈, X₇₁, and X₇₃ are F, it is expected the resulting peptide has a more desirable photoconductivity than when all of X₄₈, X₅₁, X₅₃, X₅₅, X₅₆, X₅₈, X₆₀, X₆₂, X₆₄, X₆₆, X₆₈, X₇₁, and X₇₃ are L.

In an aspect, the present disclosure provides protein fibers or peptide fibers and gels comprising those fibers. The protein fibers and peptide fibers may be referred to fibers. These fibers may comprise one or more protofibers. The one or more protofibers may comprise a plurality of proteins and/or peptides of the present disclosure.

The proteins and peptides of the present disclosure may have desirable properties. The proteins and peptides self-assemble/self-associate/aggregate to form protofibers. The assembled proteins and peptides are bound via non-covalent interactions (e.g., Coulombic interactions, hydrophobic interactions, t-t interactions, and the like, and combinations thereof) and van der Waals interactions. The protofibers may associate with other protofibers to form protein fibers or peptide fibers. The protofibers may have a diameter of less than 20 nm. The fibers may have a diameter of about 20 nm to about 2 μm, including all 0.1 nm values and ranges therebetween. The fiber has a coiled-coil morphology defining a 1-50 angstrom pore running along the length of fiber.

Without intending to be bound by any particular theory, it is considered that the peptides/proteins of the present disclosure favor longitudinal growth over lateral growth of their fibers. This may allow the fibers to achieve a higher crosslinking density within a hydrogel formed therefrom.

These protein fibers or peptide fibers may further aggregate to form an entangled network of fibers that restrict the flow of water to form a hydrogel. That is, a hydrogel of the present disclosure may comprise water and fibers of the present disclosure, where the fibers comprise protofibers comprising peptides/proteins of the present disclosure. In an embodiment, the hydrogel comprises crosslinked fibers. In an embodiment, the hydrogel comprises only non-covalently crosslinked fibers and no chemically crosslinked fibers. In an embodiment, the fibers non-covalently associate via one or more non-covalent interactions (e.g., hydrophobic interactions, π-π interactions, hydrogen bonds, and the like, and combinations thereof). Physical crosslinking is more likely to occur at low temperatures and above 1 mM protein concentrations.

A hydrogel can comprise various amounts of water. In various examples, a hydrogel comprises 80 to 99% water, such as 85 to 99.9% by weight (based on the total weight of the composition) water. In an embodiment, the hydrogel comprises about 91 to 99.9% by weight water. In various embodiments, the hydrogel comprises 99 to 99.5% weight water.

A fiber may have various compounds bound thereto. A fiber may have one or more compounds bound thereto, which may be the same or different. The compounds may be therapeutic agents. In various examples, the compound are hydrophobic. In various examples, the compounds a non-hydrophobic. In various examples, the compounds have no net charge. Non-limiting examples of compounds include dyes, antibiotics, alkaloids, vitamins, lipids, fatty acids, sugars, amino acids, phenolic compounds, extracellular materials (e.g., proteins, cells, exosomes, and the like, and combinations thereof), metals, nucleic acids, and the like, and combinations thereof.

A gel may encapsulate various compounds. A gel may encapsulate one or more compounds, which may be the same or different. The compounds may be therapeutic agents. In various examples, the compounds are hydrophobic. In various examples, the compounds a non-hydrophobic. In various examples, the compounds have no net charge. Non-limiting examples of compounds include dyes, antibiotics, alkaloids, vitamins, lipids, fatty acids, sugars, amino acids, phenolic compounds, extracellular materials (e.g., proteins, cells, exosomes, and the like, and combinations thereof), metals, nucleic acids, and the like, and combinations thereof.

The proteins or peptides or protein or peptide fibers of the present disclosure may have various desirable photoconductive characteristics. For example, they are capable of an amperage of 10⁻¹⁰ to 10⁻³ A, including all 10⁻¹² A values and ranges therebetween, under a voltage of −10 to 10 V, including all 0.01 V values and ranges therebetween.

The proteins or peptides of the present disclosure may be made by various methods known in the art. For example, the proteins of peptides of the present disclosure may be made via expression or by solid phase peptide synthesis. The proteins or peptides may further be purified and or isolated by methods known in the art (e.g., centrifugation, selective solubilization, chromatography, such as, for example, high performance liquid chromatography or other forms of liquid chromatography).

In an aspect, the present disclosure provides devices. A device may comprise a protein or peptide of the present disclosure or protein or peptide fibers of the present disclosure.

The proteins and peptides of the present disclosure may have desirable electrical and photoconductive properties. Thus, various devices requiring photoresponsiveness may comprise proteins or peptides of the present disclosure or protein or peptide fibers of the present disclosure. Examples of suitable devices include photodetectors or memristors. For example, the present disclosure may be embodied as a photodetector or memristor having a photoconductive element in electrical communication with a pair of electrodes. The photoconductive element includes proteins or peptides of the present disclosure or protein or peptide fiber(s) of the present disclosure. For example, the photoconductive element may be a medium (e.g., substrate or fluid) having the protein or peptide disposed thereon or embedded therein. In some embodiments, each electrode of the pair of electrodes is in direct electrical contact with the photoconductive element. The photodetector or memristor may have a body surrounding the photoconductive element and at least a portion of the pair of electrodes. The body may have a window such that the photoconductive element may be exposed to light. The window may be, for example, an opening in the body. The window may include a light-transmitting material (e.g., transparent, translucent, etc.) In some embodiments, the body is made from a light-transmitting material. In some embodiments wherein the photoconductive element includes a substrate, the substrate may be one of the electrodes of the pair of electrodes.

The steps of the method described in the various embodiments and examples disclosed herein are sufficient to carry out the methods of the present disclosure. Thus, in an embodiment, the method consists essentially of a combination of the steps of the methods disclosed herein. In another embodiment, the method consists of such steps.

The following Statements provide various examples of the present disclosure.

Statement 1. A protein or peptide having or comprising the following sequence:

(SEQ ID NO: 1)
VX₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃
X₂₄X₂₅X₂₆X₂₇X₂₈X₂₉X₃₀X₃₁X₃₂X₃₃X₃₄X₃₅X₃₆X₃₇,

wherein
X₁ is A, E, D, R, H, K, Q, N, or S;
X₂ is A, E, N, or Q;
X₃, X₆, X₁₀, X₁₃, X₁₇, X₂₀, X₂₄, X₂₇, X₃₁, and X₃₄ are each independently an aromatic amino acid residue, a hydrophobic amino acid residue, or a neutral amino acid residue;
X₄ is A, E, R, D, H, I, L, T, K, Q, or N;
X₅ is A, F, Q, R, K, H, D, S, or E;
X₇ is A, K, or E;
X₈ is A, K, E, D, R, H, Q, or N;
X₉ is A, T, I, L, or Q;
X₁₁ is A, E, D, H, P, I, L, K, Y, N, Q, or R;
X₁₂ is A, Q, H, E, D, K, R, or N;
X₁₄ is A, L, D, E, K, or I;
X₁₅ is A, E, D, H, Y, I, L, R, K, Q, or N;
X₁₆ is A, E, or Q;
X₁₈ is A, K, E, D, K, R, H, N, or Q;
X₁₉ is A, N, D, K, R, H, Q, or E;
X₂₁ is A, N, or Q;
X₂₂ is K, A, E, I, L, M, R, H, D, Q, N, or S;
X₂₃ is A, Q, N, I, or L;
X₂₅ is A, H, Q, R, K, D, N, Y, I, E, L, or T;
X₂₆ is A, D, E, R, K, Q, H, N, or T;
X₂₈ is A, R, E, D, K, H, N, Q, or T;
X₂₉ is A, H, E, R, D, K, I, L, N, Q, T, or Y;
X₃₀ is L, A, D, K, I, N, or Q;
X₃₂ is E, D, K, H, N, Q, A, L, R, I, or Y;
X₃₃ is A, N, Q, D, E, H, K, R, or S;
X₃₅ is S, A, P, or Q;
X₃₆ is A, K, T, D, R, H, N, Q, or E; and
X₃₇ is A, L, I, K, D, N, Q, or R. In various examples, at least one of X₃, X₆, X₁₀, X₁₃, X₁₇, X₂₀, X₂₄, X₂₇, X₃₁, and X₃₄ is an aromatic residue (e.g., F).

Statement 2. A protein or peptide according to Statement 1, wherein the aromatic amino acid residue is chosen from F, Y, and W.

Statement 3. A protein or peptide according to any one of the preceding Statements, wherein the hydrophobic amino acid residue is chosen from L, I, V, M, and A.

Statement 4. A protein or peptide according to any one of the preceding Statements, wherein the neutral amino acid residue is chosen from Q, N, T, and S.

Statement 5. A protein or peptide according to any one of the preceding Statements, wherein X₃, X₆, X₁₀, X₁₃, X₁₇, X₂₀, X₂₄, X₂₇, X₃₁, and X₃₄ are each independently F or L.

Statement 6. A protein or peptide according to Statement 5, wherein X₃, X₆, X₁₀, X₁₃, X₁₇, X₂₀, X₂₄, X₂₇, X₃₁, and X₃₄ are L.

Statement 7. A protein or peptide according to Statement 5, wherein X₃, X₆, X₁₀, X₁₃, X₁₇, X₂₀, X₂₄, X₂₇, X₃₁, and X₃₄ are F.

Statement 8. A protein or peptide according to any one of the preceding Statements, wherein the sequence is or comprises:

```
  (SEQ ID NO: 2)
VKEX₃TFX₆KNTX₁₀PQX₁₃LREX₁₇QEX₂₀NAAX₂₄QDX₂₇RELX₃₁RQX₃₄SKL;
  (SEQ ID NO: 3)
VKEX₃LFX₆KKTX₁₀EQX₁₃LEEX1₇KEX₂₀NKAX₂₄HDX₂₇RHLX₃₁ENX₃₄SKL;
  (SEQ ID NO: 4)
VKEX₃LFX₆KNTX₁₀YQX₁₃LLEX₁₇KEX₂₀NEAX₂₄YDX₂₇RHLX₃₁QQX₃₄SKL;
  (SEQ ID NO: 5)
VKEX₃TFX₆KKTX₁₀EQX₁₃AEEX₁₇KEX₂₀NKAX₂₄HDX₂₇RHAX₃₁ENX₃₄SKK;
  (SEQ ID NO: 6)
VKEX₃TFX₆KNTX₁₀EQX₁₃AEEX₁₇KEX₂₀NKAX₂₄HDX₂₇RHQX₃₁ENX₃₄SKQ;
  (SEQ ID NO: 7)
VKEX₃TFX₆KKQX₁₀EQX₁₃AEEX₁₇KEX₂₀NKAX₂₄HDX₂₇RHAX₃₁ENX₃₄SKK;
  (SEQ ID NO: 8)
VKEX₃TFX₆KKTX₁₀EQX₁₃AEEX₁₇KEX₂₀NKAX₂₄HDX₂₇RHAX₃₁ENX₃₄SKK;
  (SEQ ID NO: 9)
VSEX₃TEX₆KKTX₁₀EHX₁₃AKEX₁₇KEX₂₀NKAX₂₄HTX₂₇RHAX₃₁ENX₃₄AKN;
or
(SEQ ID NO: 10)
VKEX₃KFX₆KNTX₁₀PQX₁₃LREX₁₇QNX₂₀NMAX₂₄QDX₂₇RELX₃₁QQX₃₄STL.
```

Statement 9. A protein or peptide according to any one of Statements 1 to 7, wherein the sequence is or comprises:

(SEQ ID NO: 12)
MRGSHHHHHHGSIEGRVX₁X₂X₃X₄X₅X₆X₇X₈X₉X₁₀X₁₁X₁₂X₁₃X₁₄X₁₅X₁₆X₁₇
X₁₈X₁₉X₂₀X₂₁X₂₂X₂₃X₂₄X₂₅X₂₆X₂₇X₂₈X₂₉X₃₀X₃₁X₃₂X₃₃X₃₄X₃₅X₃₆X₃₇.

Statement 10. A protein or peptide according to Statement 9, wherein the sequence is or comprises:

```
  (SEQ ID NO: 13)
MRGSHHHHHHGSIEGRVKEX₃TFX₆KNTX₁₀PQX₁₃LREX₁₇QEX₂₀NAA
X₂₄QDX₂₇RELX₃₁RQX₃₄SKL;
  (SEQ ID NO: 14)
MRGSHHHHHHGSIEGRVKEX₃LFX₆KKTX₁₀EQX₁₃LEEX₁₇KEX₂₀NKA
X₂₄HDX₂₇RHLX₃₁ENX₃₄SKL;
  (SEQ ID NO: 15)
MRGSHHHHHHGSIEGRVKEX₃LFX₆KNTX₁₀YQX₁₃LLEX₁₇KEX₂₀NEA
X₂₄YDX₂₇RHLX₃₁QQX₃₄SKL;
  (SEQ ID NO: 16)
MRGSHHHHHHGSIEGRVKEX₃TFX₆KKTX₁₀EQX₁₃AEEX₁₇KEX₂₀NKA
X₂₄HDX₂₇RHAX₃₁ENX₃₄SKK;
  (SEQ ID NO: 17)
MRGSHHHHHHGSIEGRVKEX₃TFX₆KNTX₁₀EQX₁₃AEEX₁₇KEX₂₀NKA
X₂₄HDX₂₇RHQX₃₁ENX₃₄SKQ;
  (SEQ ID NO: 18)
MRGSHHHHHHGSIEGRVKEX₃TFX₆KKQX₁₀EQX₁₃AEEX₁₇KEX₂₀NKA
X₂₄HDX₂₇RHAX₃₁ENX₃₄SKK;
  (SEQ ID NO: 19)
MRGSHHHHHHGSIEGRVKEX₃TFX₆KKTX₁₀EQX₁₃AEEX₁₇KEX₂₀NKA
X₂₄HDX₂₇RHAX₃₁ENX₃₄SKK;
  (SEQ ID NO: 20)
MRGSHHHHHHGSIEGRVSEX₃TEX₆KKTX₁₀EHX₁₃AKEX₁₇KEX₂₀NKA
X₂₄HTX₂₇RHAX₃₁ENX₃₄AKN;
or
  (SEQ ID NO: 21)
MRGSHHHHHHGSIEGRVKEX_{3K}FX₆KNTX₁₀PQX₁₃LREX₁₇QNX₂₀NMA
X₂₄QDX₂₇RELX₃₁QQX₃₄STL.
```

Statement 11. A protein or peptide having or comprising the following sequence:

(SEQ ID NO: 22)
X₃₈TQX₃₉KNTX₄₀PQX4₁LREX₄₂QNX4₃NAAX₄₄QDX₄₅RELX₄₆QQX₄₇,

wherein X₃₈, X₃₉, X₄₀, X₄₁, X₄₂, X₄₃, X₄₄, X₄₅, X₄₆, and X₄₇ are each independently an aromatic amino acid residue, a hydrophobic amino acid residue, or a neutral amino acid residue. In various examples, at least one of X₃₈, X₃₉, X₄₀, X₄₁, X₄₂, X₄₃, X₄₄, X₄₅, X₄₆, and X₄₇ is an aromatic residue.

Statement 12. A protein or peptide according to Statement 11, wherein the aromatic amino acid residue is chosen from F, Y, and W.

Statement 13. A protein or peptide according to Statements 11 or 12, wherein the hydrophobic amino acid residue is chosen from L, I, V, M, and A.

Statement 14. A protein or peptide according to any one of Statements 11 to 13, wherein the neutral amino acid residue is chosen from Q, N, T, and S.

Statement 15. A protein or peptide according to any one of Statements 11 to 14, wherein X₃₈, X₃₉, X₄₀, X₄₁, X₄₂, X₄₃, X₄₄, X₄₅, X₄₆, and X₄₇ are each independently F or L.

Statement 16. A protein or peptide according to Statement 15, wherein X₃₈, X₃₉, X₄₀, X₄₁, X₄₂, X₄₃, X₄₄, X₄₅, X₄₆, and X₄₇ are L.

Statement 17. A protein or peptide according to Statement 15, wherein X₃₈, X₃₉, X₄₀, X₄₁, X₄₂, X₄₃, X₄₄, X₄₅, X₄₆, and X₄₇ are F.

Statement 18. A protein or peptide according to Statement 11, wherein sequence is or comprises:

MRGSHHHHHHSIEGRVKEX₃₈TQX₃₉KNTX₄₀PQX₄₁LREX₄₂QNX₄₃NAA
X₄₄QDX₄₅RELX₄₆QQX₄₇.

Statement 19. A protein or peptide having or comprising the following sequence:

MQLX₄₈X₄₉X₅₀X₅₁LX₅₂EX₅₃QX₅₄X₅₅NAAX₅₆QX₅₇X₅₈RX₅₉LX₆₀X₆₁QX₆₂VX₆₃
EX₆₄TX₆₅X₆₆KX₆₇TX₆₈X₆₉X₇₀X₇₁DX₇₂SX₇₃X₇₄X₇₅X₇₆,

wherein X₄₈, X₅₁, X₅₃, X₅₅, X₅₆, X₅₈, X₆₀, X₆₂, X₆₄, X₆₆, X₆₈, X₇₁, and X₇₃ are each independently an aromatic amino acid residue, a hydrophobic amino acid residue, or a neutral amino acid residue;
X₄₉ is P or K;
X₅₀ is Q or H;
X₅₂ is R or K;
X₅₄ is E or K;
X₅₇ is D or S;
X₅₉ is E or T;
X₆₁ is R or Q;
X₆₃ is V or E;
X₆₅ is F or Q
X₆₇ is D or E
X₆₉ is M or E;
X₇₀ is E or N;
X₇₂ is A or S
X₇₄ is K or absent;
X₇₅ is L or absent; and
X₇₆ is N or absent. In various examples, at least one of X₄₈, X₅₁, X₅₃, X₅₅, X₅₆, X₅₈, X₆₀, X₆₂, X₆₄, X₆₆, X₆₈, X₇₁, and X₇₃ is an aromatic residue.

Statement 20. A protein or peptide according to Statement 19, wherein the aromatic amino acid residue is chosen from F, Y, and W.

Statement 21. A protein or peptide according to Statement 19 or Statement 20, wherein the hydrophobic amino acid residue is chosen from L, I, V, M, and A.

Statement 22. A protein or peptide according to any one of Statements 19 to 21, wherein the neutral amino acid residue is chosen from Q, N, T, and S.

Statement 23. A protein or peptide according to any one of Statements 19 to 22, wherein X₄₈, X₅₁, X₅₃, X₅₅, X₅₆, X₅₈, X₆₀, X₆₂, X₆₄, X₆₆, X₆₈, X₇₁, and X₇₃ are each independently F or L.

Statement 24. A protein or peptide according to Statement 23, wherein X₄₈, X₅₁, X₅₃, X₅₅, X₅₆, X₅₈, X₆₀, X₆₂, X₆₄, X₆₆, X₆₈, X₇₁, and X₇₃ are L.

Statement 25. A protein or peptide according to Statement 23, wherein X₄₈, X₅₁, X₅₃, X₅₅, X₅₆, X₅₈, X₆₀, X₆₂, X₆₄, X₆₆, X₆₈, X₇₁, and X₇₃ are F.

Statement 26. A protein or peptide fiber comprising one or more protofibers comprising one or more proteins or peptides according to any one the preceding Statements.

Statement 27. A protein or peptide fiber according to Statement 26, wherein one or more compounds are bound to the protein or peptide fiber.

Statement 28. A protein or peptide fiber according to Statement 26 or Statement 27, wherein the one or more compounds are hydrophobic.

Statement 29. A protein or peptide fiber according to Statement 27, wherein the one or more compounds are dyes, antibiotics, alkaloids, lipids, fatty acids, sugars, amino acids, phenolic compounds, extracellular materials, metals, nucleic acids, and combinations thereof.

Statement 30. A protein or peptide fiber according to any one of Statements 27 to 29, wherein the protein or peptide fiber has a fiber diameter of about 20 nm to about 2 μm, including all 0.1 nm values and ranges therebetween.

Statement 31. A composition comprising a plurality of protein or peptide fibers according to Statement 26.

Statement 32. A composition according to Statement 31, wherein the plurality of protein or peptide fibers are formulated into a gel.

Statement 33. A device comprising a protein or peptide according to any one of Statements 1 to 25 or one or more protein or peptide fibers according to Statement 26.

Statement 34. A device according to Statement 33, wherein the device is photoconductive.

Statement 35. A device according to Statement 33 or Statement 34, wherein the device is a memristor.

The following examples are presented to illustrate the present disclosure. They are not intended to be limiting in any matter.

### Example 1

This example provides a description of the methods and peptides/proteins of the present disclosure.

Photoconductive nanomaterials are critical in the development of advanced optoelectronic materials, which span photovoltaics to biomedicine. These materials are often derived from inorganic crystalline semiconductors that facilitate charge transfer upon absorption of ultraviolet and infrared light. The present disclosure expands the list of materials to include single-domain protein-based nanofibers. Coiled-coil protein domains found in nature are known as electron carriers when possessing aromatic rings for pi-pi stacking and are capable of supramolecular assembly into fibers. Whereas these properties have been previously leveraged to develop conductive protein nanofibers, described herein is a conductive helical assembled fiber (CHAF) comprising aromatic residues (e.g., phenylalanines) in the pore uniquely capable of photoresponsivity. It was demonstrated that CHAF, and a leucine-replaced CHAF control (L-CHAF) exhibited unique photoconductive responses when exposed to near infrared (NIR) light. The sequence-controlled system reported here illustrates conductive protein fibers capable of photoresponsivity as a promising platform for the generation of new biocompatible nanomaterials.

Described herein is a conductive helical assembled fiber (CHAF), where protein photoconductivity is directly dependent on intensity of near-infrared (NIR) light. CHAF is designed as a conductive coiled-coil protein akin to engineered G. sulfureduccens pili and other inspired coiled-coils. The coiled-coil core is lined with an aromatic residue, phenylalanine, in the a and d helical wheel positions. A leucine-replaced conductive helical assembled fiber (L-CHAF) in which the core is lined with leucine in place of the phenylalanines is also explored. Uniquely, CHAF and L-CHAF are full-length recombinantly expressed proteins based on coiled-coil electrostatics that allows for the control of the extent of fiber assembly and is capable of conductivity. Interestingly, conductivity is not dependent on the presence of aromatic residues (e.g., phenylalanines). Rather, the presence of aromatic residues (e.g., phenylalanines) vs. aliphatic residues (e.g., leucines) appears to promote conformational changes in the coiled-coil fibers that produce different conductivities. Furthermore, there is an inherent difference in the ability of CHAF to produce reliable photoconductivity where L-CHAF cannot.

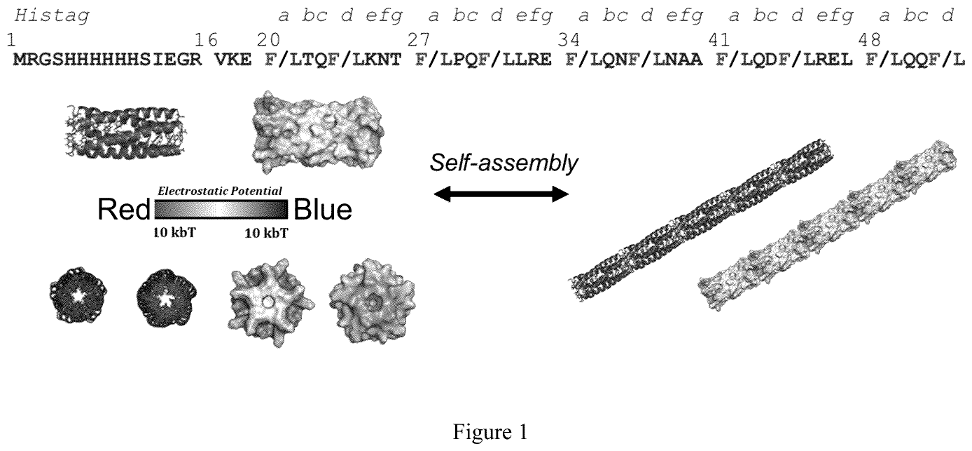

Design. Described is a coiled-coil capable of selective end-to-end and longitudinal assembly to study pi-pi interactions along thin fibers (FIG. 1). The protein sequence Q, derived from the homopentameric coiled-coil domain of the cartilage oligomeric matrix protein (COMPcc), has been used to produce large nanofibers. Using the Q sequence as a starting design, phenylalanines were introduced in the a and d helical wheel positions of the coiled-coil, resulting in an approximate separation of 5 Å (measured using the distance function in PyMOL) between the center of aromatic sidechains, similar to the pi-pi interaction distance previously estimated in cyclic voltammetry experiments.

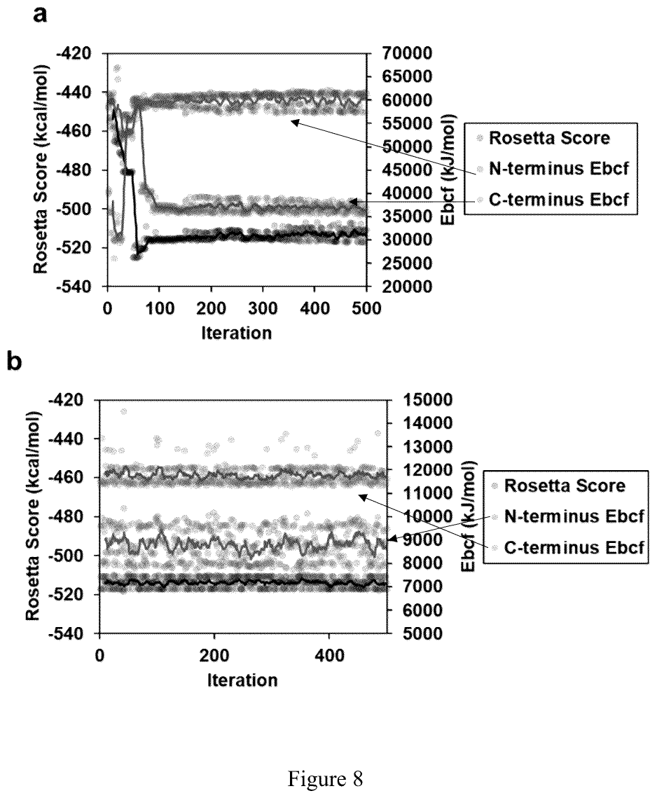

To promote C-termini phenylalanine interactions with a neighbouring coil-coil upon end-to-end stacking, the final three residues following the final d position were removed (residues in the e, f g position). Recently, it was demonstrated that lowering the electrostatic potential of coiled-coil fibers reduced lateral assembly to produce thinner fibers. Therefore, after replacing all the a and d positions with phenylalanines in the Q sequence, a single chain was subjected to a Monte-Carlo search interfacing with Rosetta and PDB2PQR-APBS electrostatics software to remove electrostatic potential of the coiled-coil protein while maintaining or improving the Rosetta score via iterative mutations of solvent-exposed residues (aka those in the b, c, and f positions) (FIG. 8).

A search was used to run through 500 mutations (FIG. 8a). A single chain from the resulting low-electrostatic potential coiled-coil was then submitted into a similar Monte-Carlo search to increase the Rosetta score and instead selectively increase the electrostatic potential at the first five residues (or 2 solvent-exposed, mutable residues) of the N- and C-termini to improve the chance of end-to-end stacking and promote the supramolecular assembly of thin nanofibers (FIG. 8b). The final protein sequence chosen was based on Rosetta score, low E_{bcf} for the N- and C-terminus, and if the electropotential map exhibited positive and negative patches near the ends of the coiled-coil. The protein sequence was then symmetrically relaxed using Rosetta with a final score calculated to be −447 kcal/mol. The resulting electrostatic potential map confirmed the presence of very localized positive and negative patches at the termini while maintaining the coiled-coil structure with conductive phenylalanines in the pore. To assess the importance of phenylalanines in the pore, a leucine-replaced conductive helical assembled fiber (L-CHAF) was designed with leucines in the a and d helical wheel positions instead.

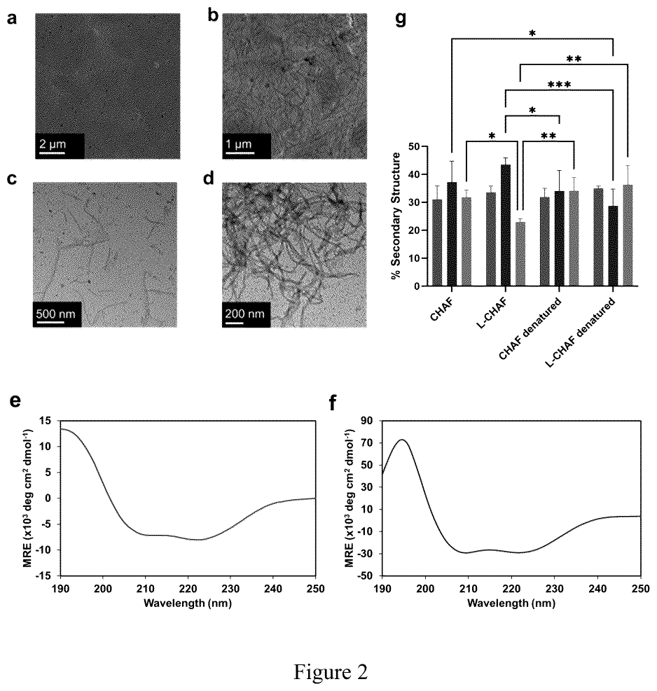

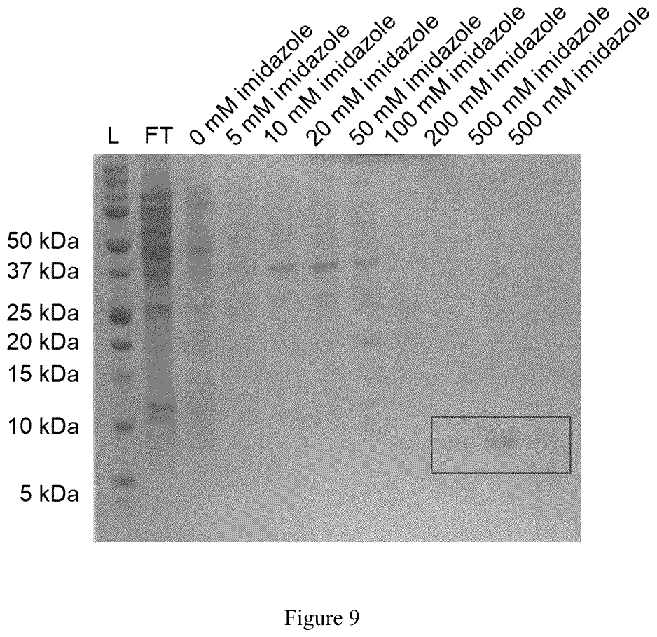

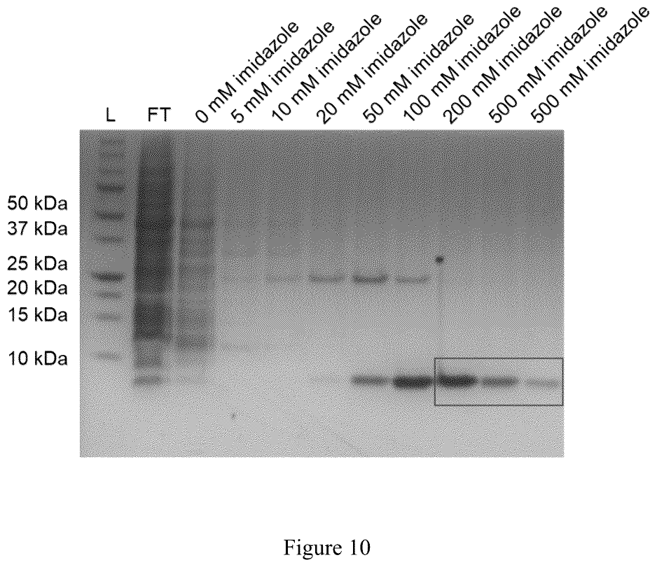

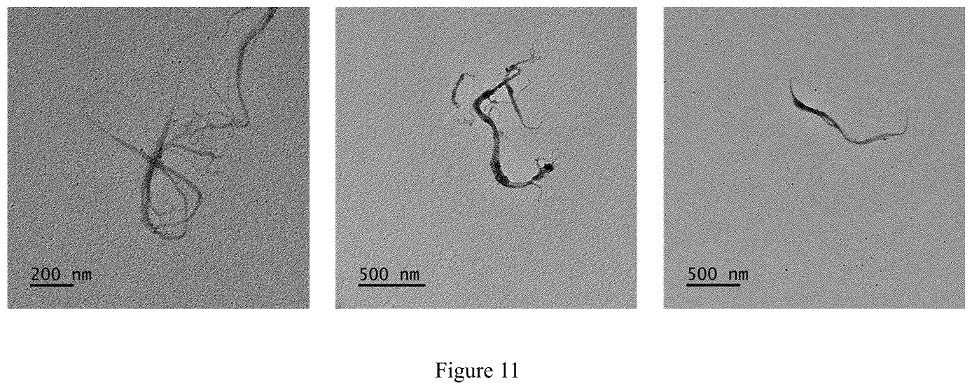

Nanofiber Assembly and Structure. CHAF and L-CHAF were both expressed and purified successfully (FIGS. 9-10) as has been done previously for our coiled-coil protein fiber system. Scanning electron microscopy (SEM) and transmission electron microscopy (TEM) images confirmed the presence of nanofibers and nanofiber bundles in CHAF (FIG. 2a-d). Nanofibers possessed an average diameter of 24.1±6.2 nm for CHAF and 23.8±5.2 nm for L-CHAF by TEM (FIG. 11). Apropos to the design, these fibers rank as the thinnest fibers we have designed, demonstrating the ability to tune the fibers for less lateral assembly. Previously, COMPcc had shown the smallest diameter fibers at 47±22 nm.

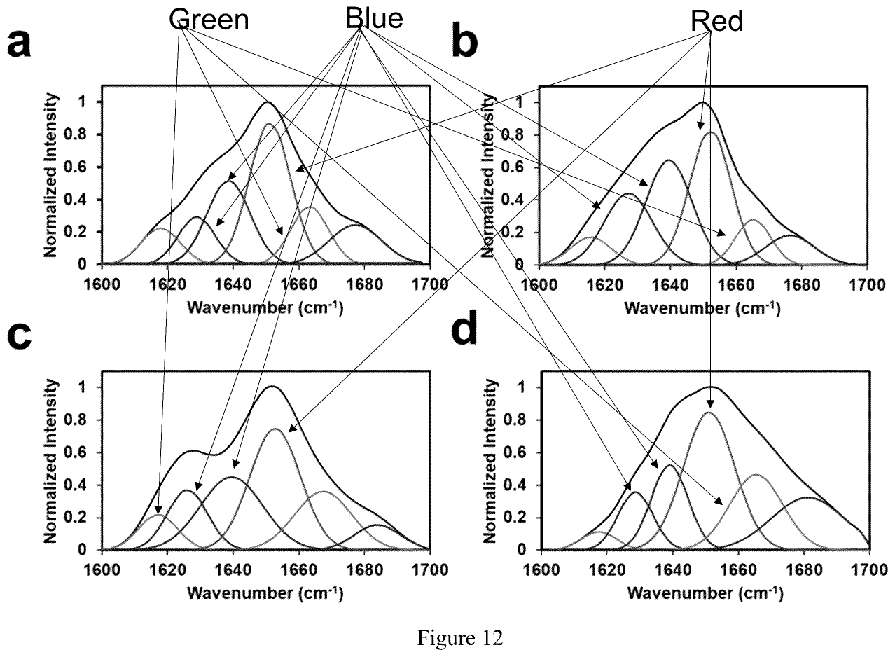

The secondary structure of CHAF and L-CHAF at representative concentrations were assessed by circular dichroism (CD) (FIG. 2e-f) and attenuated total reflection-Fourier transform infrared (ATR-FTIR) spectrometry measurements (FIG. 2g, FIG. 12a-d) using 500 μM samples. Analysis of the spectra by CD (FIG. 2e-f, Table 1) exhibited stark differences in helical content of CHAF and L-CHAF. Using ATR-FTIR after thermal challenge (FIG. 2g, Table 2), it was further demonstrated that CHAF and L-CHAF possessed differences in thermodynamic stability.

By CD, CHAF revealed an expected double minima of −6,600±1,100 deg·cm²·dmol⁻¹ at 208 nm and −8,000±2,000 deg·cm²·dmol⁻¹ at 222 nm, where L-CHAF demonstrated a double minima of −24,600±1,300 deg·cm²·dmol⁻¹ at 208 nm and −25,200±2,000 deg·cm²·dmol⁻¹ at 222 nm, similar to other coiled-coil proteins. Moreover, this illustrates a 3-fold increase in magnitude for L-CHAF as compared to CHAF. As a result, deconvolution of secondary structure by BestSel demonstrated that CHAF possessed 13.4±2.9% α-helical, 32.9±13.7% β-sheet, and 53.7±3.2% random coil content whereas L-CHAF possessed 91.0±4.5% α-helical, 0.0±0.0% β-sheet, and 9.0±4.5% random coil content. Both proteins displayed 222/208 ratios >1 (Table 1), indicative of strong coiled-coil content, however, L-CHAF exhibited a clear increase in α-helicity. This was likely due to the bulky phenylalanines in the hydrophobic pore of CHAF disrupting helical structure, similar to secondary structure signal of parent protein, Q, which possessed the central, rigid residue P28 that was indicative of a less structured protein.

To determine the thermostability of the CHAF and L-CHAF protein, temperature scans were performed from 20° C. to 90° C. The CHAF protein possessed a melting temperature (Tₘ) of 54.9±1.8° C. (Table 1) in 50 mM Na₂HPO₄ pH 4.0 buffer similar to previous protein fibers designed from COMPcc and Q previously, which ranged from 37-61° C. This Tₘ was used to assess the impact of protein annealing on the photoconductivity of CHAF devices. In comparison, L-CHAF, exhibits a melting temperature of 75.8±9.2° C. (Table 1) in 50 mM Na₂HPO₄ pH 4.0 buffer revealing a significant increase in thermostability (p-value 0.02). The 20° C. increase in thermostability could be attributed to the stabilizing properties of leucine in the pore of a coiled-coil as opposed to phenylalanines.

Consistent with CD, ATR-FTIR demonstrated CHAF possessed increased random coil content at the expense of β-sheet secondary structure compared to L-CHAF (FIG. 2g). However, ATR-FTIR exhibited that CHAF and L-CHAF revealed similar structured content (α-helical and β-sheet) by deconvolution of the ATR-FTIR peaks (FIG. 12a-d, Table 2), both possessing 31-34% helical content.

To explore the impact of temperature on secondary structure of CHAF and L-CHAF at high temperatures, ATR-FTIR was performed again after 10 min incubation of CHAF samples at its Tₘ of 55° C. (FIG. 2g, FIG. 12c) and 10 min of incubation of L-CHAF samples at its Tₘ of 76° C. (FIG. 2g, FIG. 12d). Denatured CHAF possessed 31.8±3.2% α-helical, 34.0±7.4% β-sheet, and 34.1±4.7% random coil content while denatured L-CHAF possessed 34.9±0.9% α-helical, 28.7±6.0% β-sheet, and 36.3±6.9% random coil content. Overall, L-CHAF demonstrated a significant loss in β-sheet content with an increase in random coil content, whereas the secondary structure of CHAF remained mostly similar before and after annealing (FIG. 2g)

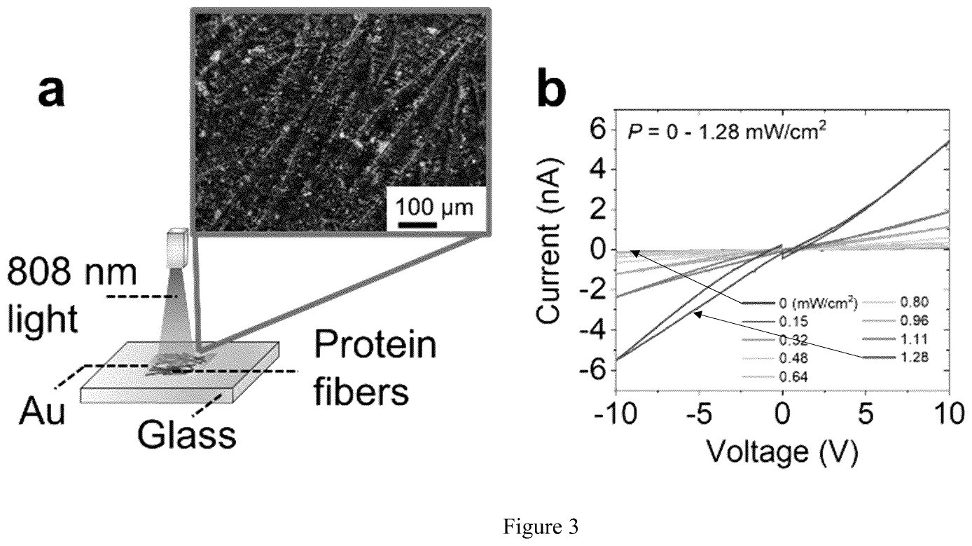

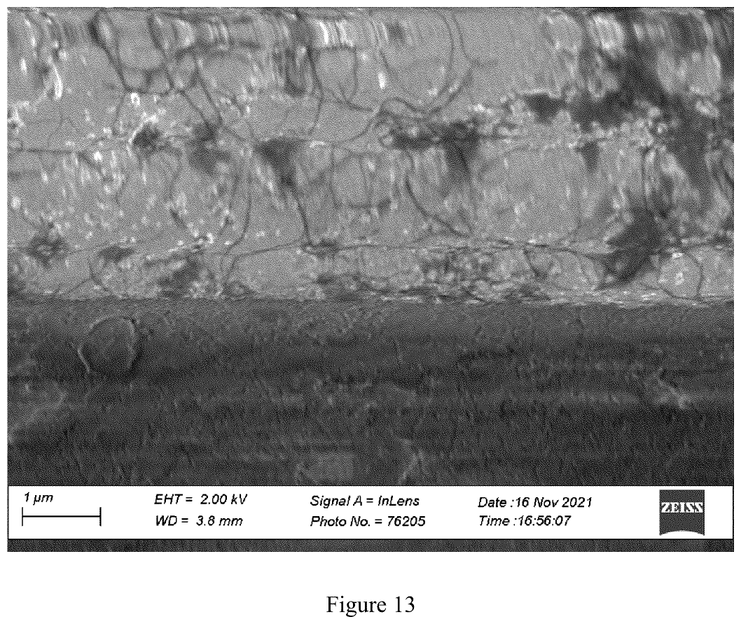

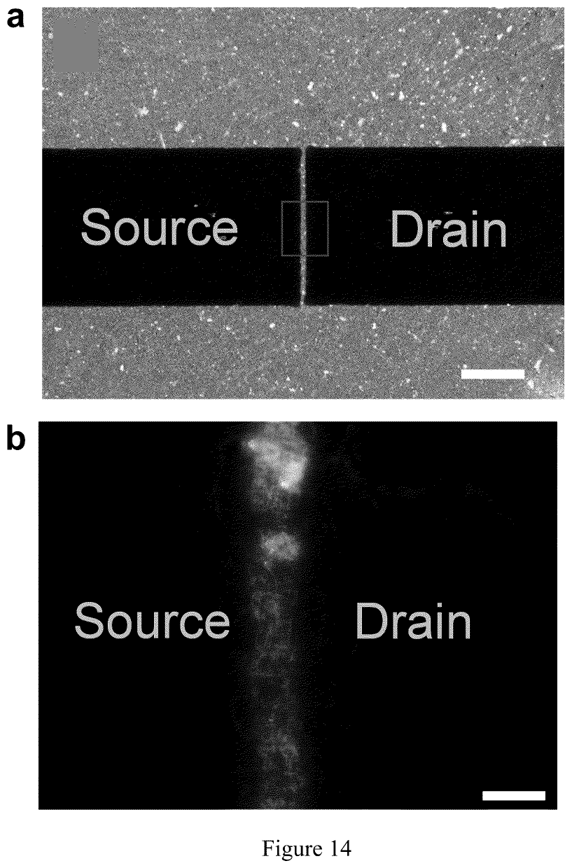

Photoconductivity. Photodetectors were fabricated by drop casting CHAF protein fibers from a 1.5 mM solution in water with 50 mM Na₂HPO₄ pH 4.0 onto cleaned glass substrates with thermally evaporated co-planar gold electrodes (FIG. 3a, FIG. 13, FIG. 14). A polarized optical micrograph (POM) of CHAF revealed the presence of long, thin fibers with diameters ˜30 nm surrounded by birefringent salt crystals (Inset of FIG. 3a).

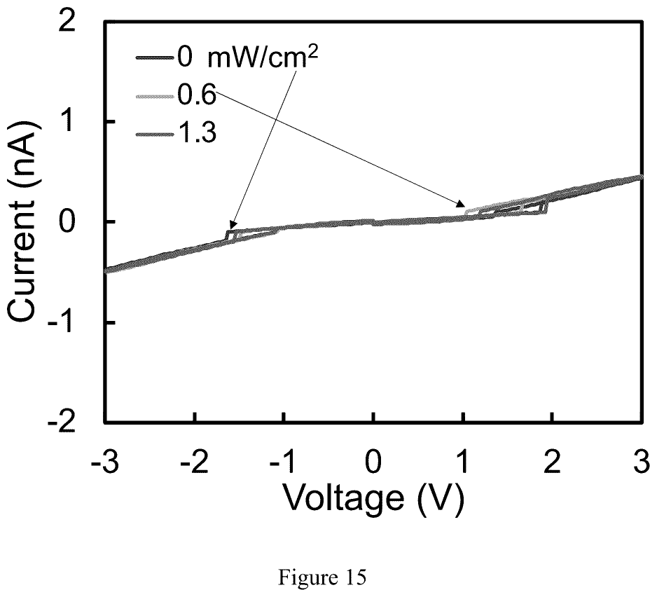

Current vs voltage (I-V) curves of CHAF photodetectors as a function of incident light intensity (λₑₓ=808 nm) are displayed in FIG. 3b. At applied voltages between −10 to 10 V, current levels on the order of 10⁻¹⁰ A were measured in the dark. In the presence of 808 nm-light, current levels increase by 1-2 orders of magnitude, reaching a maximum of 5.2 nA at an applied bias of 10 V and a light intensity of 1.28 mW/cm². In comparison, no photoresponse was detected using an excitation wavelength of 365 nm (FIG. 15). For reference, devices comprising only NaCl exhibited current levels between 10⁻² and 10⁻¹¹ A and no photoresponse.

Protein conductivity in the dark state has been established previously via electron transfer or electron transport where electrons are exchanged between an ionically conductive electrolyte in contact with a protein or through a difference in electron potential between two electrodes, respectively. This system relies on the latter.

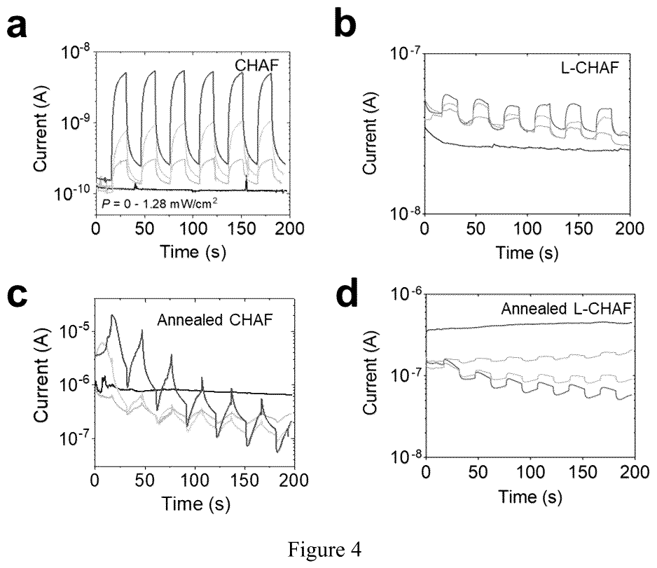

To assess the differences of protein structure on observed conductivity and photoresponse, photodetectors comprising CHAF fibers, L-CHAF fibers, annealed CHAF fibers, and annealed L-CHAF fibers were compared. Time-dependent photoresponse of the three devices was monitored while the incident light was alternately turned on and off in 25 s intervals (FIG. 4). A strong relative photoresponse was measured for CHAF photodetectors, with photocurrent increasing upwards of two orders of magnitude from baseline with increasing light intensity. The maximum and minimum photocurrents during illumination were steady from cycle to cycle. Transient effects were apparent in the relatively slow rise and fall times upon turning the light on and off, suggesting the presence of charge traps in the fibers. Surprisingly, the dark current of L-CHAF was three orders of magnitude higher than that of CHAF photodetectors despite the absence of phenylalanines that may cause pi-pi stacking in the coiled-coil core (FIG. 4b). On the other hand, rise and fall of current with light was relatively lower when normalized to the baseline indicating less relative light responsivity.

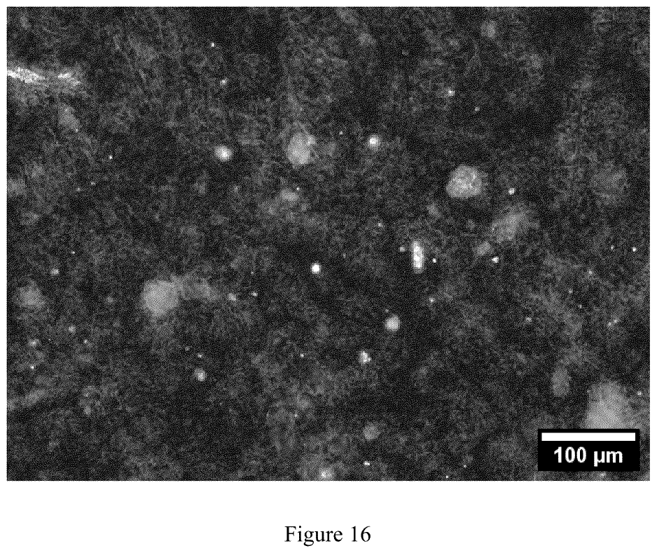

It was expected that the photoresponse may also be the result of conformational changes in the protein structure. Thus, the role of bundle morphology on conductivity and photoconductivity was further examined by incubating CHAF and L-CHAF devices at respective protein Tₘ. POM images revealed that large fiber bundles broke apart to form a random, interconnected network of thin hair-like fibers during thermal annealing (FIG. 16). Annealing is expected to disrupt fiber stacking by unravelling and repacking the protein. The time-dependent photoresponse of a CHAF photodetector after annealing at 55° C. for 10 min and of a L-CHAF photodetector after annealing at 76° C. for 10 min is displayed in FIG. 4c and FIG. 4d, respectively. Interestingly, current levels measured for annealed CHAF photodetectors exhibited the highest dark current of 10⁻⁶ A among the four devices. Some photoresponse was also observed, but with slower rise and fall times. Since some photoresponse was also present in L-CHAF, it was concluded that aromatic residues, such as, for example, phenylalanines, in the CHAF protein may provide increased photoresponse while providing increased random coil structure (FIG. 2e-g) due to bulkiness in the pore. Conversely, increased structure of L-CHAF may allow for increased conductivity through the protein backbone indicated by lower random coil content (FIG. 2e-g, Table 1, Table 2). In comparison, ATR-FTIR measurements suggested no loss of helical content after thermal challenge for CHAF and L-CHAF. Interestingly, dark current levels rise in both annealed CHAF and L-CHAF devices where it was hypothesized that annealed devices allow for separation and repacking of the protein. These results suggested that retention of helicity in CHAF and L-CHAF (FIG. 2g) and the presence of thin fibers (FIG. 16) are most critical for conductance. The interconnected network of thinner fibers may also allow for increased contacts for current transfer. L-CHAF devices after annealing also exhibit a decreasing trend in relative conductivity with time, consistent with CHAF devices after heating, suggesting a loss of protein fiber stability after exposure to high heat. It has also been established that changes in conformation of supramolecular assembling semiconducting polymers influence their ability to form tie-molecules that allow for charge transfer.

Excitation Mechanism. Differences in the photoresponse are likely also linked to conformational changes in the protein structure. Because absorbance of light at 808 nm of the protein was not observed, it was hypothesized that excimers are being formed beyond the localized transitions of aromatic sidechains of phenylalanines in the case of CHAF or histidines in the case of CHAF and L-CHAF. Histidines also would allow for similar pi-pi stacking between imidazole side chains, where pi-pi stacking has been previously established for imidazole dimer units. The excimer formation in aromatic molecules is studied in more detail in prior studies in model dimeric-benzene systems.

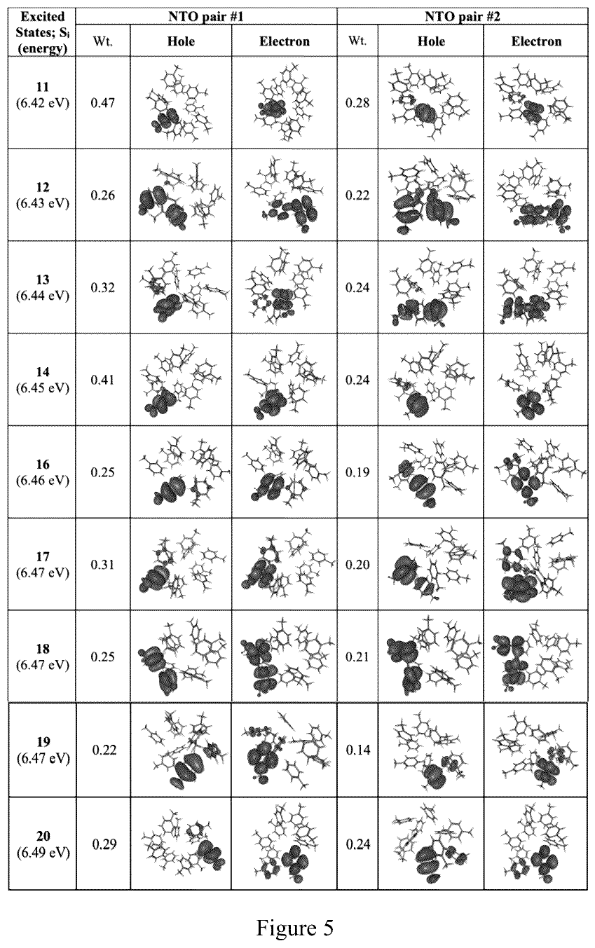

It was hypothesized that the first few bright S₀→Sᵢ transitions are due to local pi-pi* type excitations, while Sᵢ→Sₓ transitions are the result of excimer (Sₓ) formation. The Sₓ states were much higher in energy with respect to the S₀ state. Note that the Sᵢ states are predominantly localized on one Phe sidechain with only a few states where the excitation is shared on two Phe sidechains (FIG. 5). To explain this photoexcitation of aromatic rings involving a pi-pi* transition, time-dependent density functional theory (TDDFT) was employed. The initial structure used in the TDDFT calculations is obtained from a 1-μs molecular dynamics (MD) simulations. It was found that the states Sᵢ=11 to Sᵢ=20 possessed excitation energy of 6.4-6.5 eV (FIG. 5). Except for Sᵢ=15, all other states from Sᵢ=11 to Sᵢ=20 were bright, with oscillator strength values ranging from 0.01 to 0.07. The natural transition orbitals (NTOs) of corresponding transitions were localized over one or two aromatic rings. Next, the Sᵢ→Sₓ transitions were investigated that were in the 1.4-1.5 eV range since it corresponded to −800 nm peak in absorption spectra. It was found that Sₓ states were achieved when x=172 to 200, where it was considered only Sₓ states within 0.1 eV of one another. Subsequently, the integrated oscillator strength (fᵢₙₜ) was also calculated by summing over oscillator strengths of all possible Sᵢ→Sₓ transitions to account for intensity sharing between closely-spaced states. This resulted in a calculated fᵢₙₜ value of 0.039, which indicated an overall bright Sᵢ→Sₓ transition.

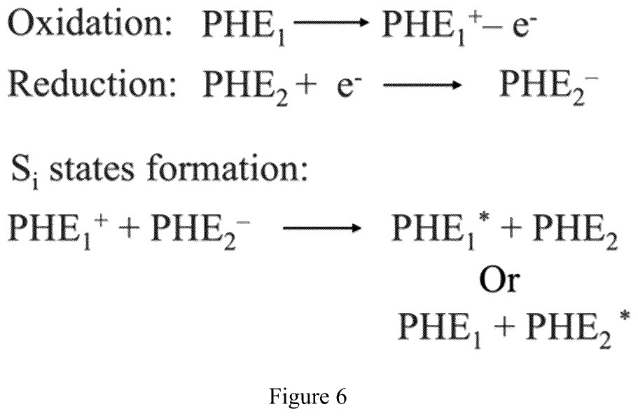

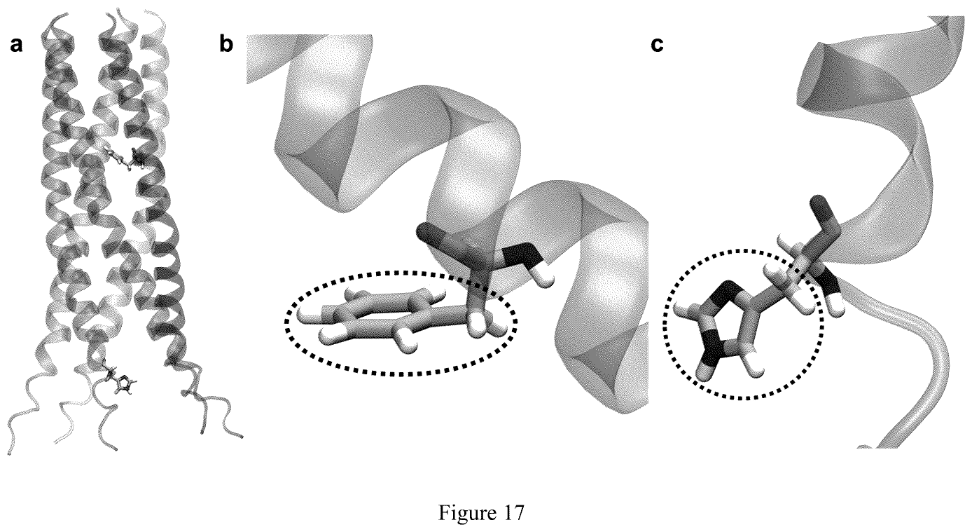

It was also hypothesized that the Sᵢ states are generated through electrochemical reactions. Similar states are often created under applied voltage in the area of electrochemiluminescence. Under that premise, one redox-active sidechain can get oxidized and reduced at two electrodes, and they react to form an excited state. This scheme is described in FIG. 6. Therefore, the observed current in L-CHAF originates from the His residues in the tag. The redox potential for one of the phenylalanines (PHEs) in the middle of the assembly and one of the first histidines from one of the chains of CHAF (Table 3, FIG. 17) was calculated. The redox potentials of phenylalanine and histidine residues are very similar in CHAF. Therefore, the applied voltage should be able to oxidize and reduce histidine akin to the redox behavior of phenylalanine in CHAF.

In summary, described is the development of a protein-based conductive nanofiber, CHAF, comprised of a single coiled-coil domain capable of photoresponsivity. In comparison to a leucine control, L-CHAF, it was established that aromatic residues (e.g., phenylalanine residues) in the pore of the coiled-coil allow for an increase in favorable sidechain interactions responsible for reproducible photoresponse whereas increased structure in the protein via L-CHAF allows for increased conductivity. Similarly, photodevices annealed for 10 min at the protein Tₘ exhibits increases in overall conductance and a relative loss in photoresponse suggesting protein fiber photoconductivity may be tuned through annealing. Photodevices drop cast with CHAF fibers demonstrate photoconduction when exposed to 808 nm incident light on the order of 10⁻¹⁰ to 10⁻⁹ A at 1.28 mW/cm². In contrast, the more denatured CHAF and L-CHAF fibers exhibit a decreased relative rise and fall in photoresponse and increased conductivity on the order of 10⁻⁶ A at 1.28 mW/cm². It is hypothesized that the mechanism for CHAF and L-CHAF with excimer formation through the aromatic amino acid sidechains. Furthermore, it was established that coiled-coil supramolecular assembly can also be computationally controlled through sequence alone, suggesting these single-domain protein materials offer a unique path toward tunable photoconductive materials. These properties can be employed to develop protein-based photoconductive biomaterials for a large variety of fields, including photovoltaics and biomedicine.

Materials. Chemically competent M15MA E. coli cells were gifted from David Tirrell at California Institute of Technology. Bacto-tryptone, sodium chloride (NaCl), yeast extract, tryptic soy agar, ampicillin sodium salt, sodium phosphate dibasic anhydrous (Na₂HPO₄), sodium hydroxide (NaOH), dextrose monohydrate (D-glucose), magnesium sulfate (MgSO₄), calcium chloride (CaCl₂)), manganese chloride tetrahydrate (MnCl₂·4H₂O), urea, cobaltous chloride hexahydrate (CoCl₂·6H₂O), isopropyl β-D-1-thiogalactopyranoside (IPTG), Pierce bicinchoninic acid (BCA) assay kit, Pierce snakeskin dialysis tubing 3.5 K molecular weight cutoff (MWCO), sodium dodecyl sulfate (SDS). The twenty naturally occurring amino acids, dimethylsulfoxide (DMSO), thiamine hydrochloride (vitamin B), thioflavin T (ThT) were purchased from Sigma Aldrich. Hydrochloric acid (HCl), Coomassie® Brilliant Blue G-250 were purchased from VWR. HiTrap FF 5 mL columns for protein purification were purchased from Cytiva Life Sciences. Macrosep and Microsep Advance Centrifugal Devices 3K MWCO and 0.2 μm syringe filters were purchased from PALL. Acrylamide/bis solution (30%) 29:1, and natural polypeptide sodium dodecyl sulfate-polyacrylamide gel electrophoresis (SDS-PAGE) standard were purchased from Bio-Rad. Copper (II) chloride anhydrous (CuCl2), sodium selenite (Na₂SeO₃), and imidazole were purchased from Acros Organics. Formvar/carbon-coated copper grids (FCF400-Cu) and 1% uranyl acetate for transmission electron microscopy were purchased from Electron Microscopy Sciences. Glass slides for photodetector platforms were purchased from Fisher Scientific. Chromium (99.95% pure) and gold (99.99% pure) for photodetector electrodes were purchased from RD Mathis and Angstrom Engineering, respectively, and used as received.

Methods. Computational Modeling and Calculation of Electrostatics: Rosetta suite of macromolecular modeling tools (Version 3.5) was used to model protein mutants and calculate Rosetta scores. The Rosetta Relax protocol was used on protein sequences using the symmetry of COMPcc (PDB: 3V2P) with the all-atom energy score function. To calculate the electrostatic potential of the termini of coiled-coil protein variants, PDB2QR and APBS was used as described previously. In the effort to add and remove electrostatic potential with consideration of mutations only to the coiled-coil, the sequence length, l, of the coiled-coil without the His-tag was used, 35 residues in length. The electrostatic potential of the residues in the b, c, and f helical wheel positions (E_{bcf}) of the N- and C-termini were calculated for the first 17 residues of each half in the first Monte-Carlo search to remove electrostatic potential and for the first 3 residues of each half in the second Monte-Carlo search to add small electrostatic potential patches at each terminus. A trimodal Monte-Carlo search was used based on the combined probabilities of an improved: 1) Rosetta Score, 2) E_{bcf} at the N-terminus, and 3) E_{bcf} at the N-terminus. The Monte-Carlo search used Equation 1 to determine the probability of selecting a mutant with a worse Rosetta Score (P_{RS}).

$${P}_{RS} = {e}^{\frac{( {RS}_{current} - {RS}_{previous} )}{RT \times C \times {RS}_{previous}}} \qquad Equation 1$$

Similarly, Equation 2 was used to determine the probability of selecting a worse E_{bcf} (P_{Ebcf}) at the N- or C-terminus.

$${P}_{Ebcf} = {e}^{\frac{- \vert ( {Ebcf}_{current} - {Ebcf}_{previous} ) \vert}{RT \times C \times {Ebcf}_{previous}}} \qquad Equation 2$$

RS is the Rosetta score, RT is the molar gas constant at room temperature, and C is an empirical constant used to constrain the probability criteria during the search (a C of 360 and 500 was used in Equation 1 and Equation 2, respectively, in the final search for CHAF). Final protein structure was visualized using PyMOL.

CHAF and L-CHAF Expression: CHAF and L-CHAF protein were expressed as described previously. Briefly, pQE60/CHAF and pQE60/L-CHAF plasmids were each cloned and purchased from Genscript. Plasmids were transformed into chemically competent M15MA E. coli cells on tryptic soy agar plates. Colonies were inoculated in supplemented M9 media and allowed to grow to an optical density at 600 nm (OD₆₀₀) of 0.8-1.0 before induction with 200 μg/mL IPTG. Cells were then incubated for 3 hours at 37° C. and 350 rpm before harvesting by centrifugation at 5,000×g at 4° C. for 30 minutes in an Avanti J-25 centrifuge (Beckman Coulter) and stored at −20° C. until purification. 12% SDS-PAGE was used to confirm expression.

CHAF and L-CHAF Purification: CHAF and L-CHAF protein were purified by affinity chromatography using a cobalt-charged HiTrap IMAC FF 5 mL column. First, CHAF and L-CHAF expression pellets were reconstituted using 40 mL Buffer A (50 mM Tris-HCl, 500 mM NaCl, 6 M urea, pH 8.0). 20 mL of reconstituted cells were then lysed using a Q500 probe sonicator (QSonica) at 55% amplitude for a total of 2 minutes for 5 s on and 5 s off. The lysed cells were then centrifuged at 11,000×g for 50 minutes. HiTrap columns were equilibrated with Buffer A and lysate was flown through at 1 mL/min. Protein was then eluted by increasing concentrations (0-100%) of Buffer B (50 mM Tris-HCl, 500 mM NaCl, 6 M urea, 500 mM imidazole). Pure fractions were assessed by 12% SDS-PAGE and dialyzed using a stepwise decrease in urea (three 5 L buckets at 3 M, 1.5 M, and 0.75 M urea respectively) followed by five consecutive 5 L buckets all containing 50 mM Na₂HPO₄ pH 4.0. Protein was then concentrated using 3 kDa MWCO Macrosep and Microsep Advance centrifugal devices (Pall Corporation) to 1.5 mM. Protein concentration was determined by bicinchoninic acid (BCA) assay with a standard curve made using dilutions of bovine serum albumin (BSA).

Circular Dichroism Spectroscopy: CHAF and L-CHAF protein secondary structure was assessed at 10-15 μM using a Jasco J-815 circular dichroism (CD) spectrometer with a PTC-423S single position Peltier temperature control system. Temperature scans were performed from 20° C. to 90° C. at 1° C. step sizes measured at 222 nm. Wavelength scans were performed from 195 to 250 nm at 1 nm step sizes in 50 mM Na₂HPO₄ pH 8.0 buffer. Mean residue ellipticity (MRE) was calculated as described in previous studies. Secondary structure content was predicted by BestSel software.

Attenuated Total Reflectance-Fourier Transform Infrared Spectroscopy: CHAF and L-CHAF secondary structure was assessed by peak deconvolution of attenuated total reflectance-Fourier transform infrared (ATR-FTIR) spectroscopy. ATR-FTIR measurements were performed using a Nicolet 6700 Fourier Transform Infrared Spectrometer equipped with a diamond ATR accessory and a mercury cadmium telluride (MCT)-A detector. Spectra were collected for 5 μL of 500 μM protein from 4000-400 cm⁻¹ with 4.0 cm⁻¹ increments and normalized using buffer background and analyzed from 1700-1600 cm⁻¹ corresponding to the amide I region at RT. Peaks were deconvoluted using Gaussian functions in PeakFit software until the goodness of fit reached r²≥0.99.

Scanning Electron Microscopy: Scanning electron microscopy (SEM) images were taken with a Carl-Zeiss field emission scanning electron microscope after drop casting 400 μM of CHAF protein in 50 mM Na₂HPO₄ pH 4.0 buffer on a silicon wafer at RT.^{[42]} Images were also taken after drop casting 4 μM protein 50 mM Na₂HPO₄ pH 4.0 buffer onto Au electrode photodetector devices.

Transmission Electron Microscopy: Transmission electron microscopy (TEM) images were taken with a FEI Talos L120C TEM. Samples were diluted to 50 μM and 3 μL was spotted on Formvar/carbon-coated copper grids followed by a 5 μL wash with water, and 3 μL staining with 1% v/v uranyl acetate solution each with incubation times of 1 min. Between steps, filter paper was used to wick the grids. Following imaging at RT, fibrils were sized in ImageJ software (Version 1.52q).

IR Photodetector Fabrication: Glass substrates were sequentially cleaned with acetone, ethyl alcohol, and deionized water for 15 min each and then exposed to UV ozone for 10 min. Next, 20 μL of solution for CHAF and L-CHAF fibers from 1.5 mM solution in water with 50 mM Na₂HPO₄ (pH 4.0) were drop cast onto the glass substrates at 25° C. in a nitrogen-filled glove box. These films were then dried in the glove box for 1 h at room temperature. 10 nm-Cr/100 nm-Au electrodes were thermally evaporated at 0.05 nm s⁻¹ onto the dried films at a pressure of 1×10⁻⁶-5×10⁻⁶ mbar. Electrode areas were 0.000187 cm², as defined by a shadow mask during the metal deposition.

Photoresponse Measurements: Photodetector I-V curves were collected at room temperature with a digital source meter (Keithley model 2636B). The applied voltage was swept from 0 to 10 V, then to −10 V and back to 0 V at a scan speed of 50 mV s⁻¹ (20 mV step width with 60 ms duration per step). An 808 nm laser diode (IRM808TA-200FC, SLOC lasers) was used as a light source. The light intensity was measured using a power meter (Model 843-R, Newport). An attenuator was also employed to tune the irradiation power. Time-dependent photoresponse was measured by repeatedly turning on and off light irradiation with intensities ranging from 0 to 1.28 mW cm⁻² in 15 s increments while measuring current flow at a fixed bias of 10 V.

Molecular Dynamics and Excitation Energy Calculations: CHAF protein pdb files from Rosetta were first equilibrated for 2 ns while keeping the heavy atoms of the proteins fixed, followed by another 2 ns of equilibration, where everything could relax. 1-μs MD simulation was performed using CHARMM36m force field parameters and the TIP3P water model. Note that these simulations do not include the His tags. The simulations were performed using the NAMD package. The final structure from the 1 μs MD simulation was used for creating a cluster model. In the cluster model, the side chains of two phenylalanines (residues 34 and 37) from each of the five chains were included. The valency of the beta carbons for each of the phenylalanine residues was satisfied by adding a hydrogen atom. Therefore, the cluster model contains ten toluene molecules. The geometry of the cluster at CAM-B3LYP/6-31G* level of theory was optimized while fixing the position of the beta carbon atoms for each phenylalanine residue. Using the ground state optimized geometry, TDDFT (Time-dependent density functional theory) excitation energies for 200 excited states at the same level of theory was calculated. The CPCM implicit solvent model was used to mimic the protein environment by a solvent with low dielectric constant (ε=6.2528). These TDDFT calculations and the following QM/MM calculations are performed using QChem.

To calculate the redox potential calculation of PHE and HIS residue, linear response approximation (LRA) was used. First, ensemble sampling from MD simulation was generated followed by QM/MM single-point energy calculation to obtain vertical energy gaps (VEGs) between oxidized and reduced forms; <VEG>=<E(ox:Phe⁺)−E(red:Phe)>, where E is the energy of the redox-active sidechain. One PHE residue from the middle of CHAF and one HIS residue closer to the center of CHAF was chosen (FIG. 17). Initial force field parameters for the reduced state of the Phe and His sidechains were taken from CHARMM36m. Then, the geometry of PHE and HIS was optimized by wb97xd/6-31+G* level of theory, and NBO charges were calculated at the optimized geometry. Geometry optimization and NBO calculation were performed in acetonitrile solvent to be consistent with TDDFT calculations and mimic the protein environment. Partial charges in the force field parameters were replaced by NBO charges for both oxidized and reduced states, and the rest of the parameters remained the same for both states, as in CHARMM36m. The CHAF system (with the His tags) with five monomers was prepared in a 79×76×116 A³ water box. First, a constrained 2 ns equilibrium simulation was performed to equilibrate solvent density, followed by a 10 ns equilibrium simulation where everything was allowed to relax. Then, a 1-pts MD simulation with 2 fs timestep and under 1 atm pressure and 310 K temperature to obtain an equilibrated assembly structure. In these simulations, CHARMM36m force field parameters for protein residues and the TIP3P water model was used. Then, an additional 3 ns MD simulation was initiated, which corresponded to oxidized and reduced states of Phe and His sidechains from the last step of the 1-μs MD simulation. CHARMM36m force field parameters were used for the rest of the protein. QM/MM single-point energy calculations was performed for 100 frames collected from the last 2 ns of the specific oxidation state simulations. Single-point VEG calculations were performed at wb97xd/6-31+G* level of theory with side chain of the selected Phe and His residues were included in the QM region (FIG. 17). A link atom (H) was used to satisfy the valency of the QM region after breaking the QM-MM bond, followed by the redistributed charge and dipole (RCD) method to treat the QM/MM interface. The rest of the protein, water, and ions were included as MM point charges with electrostatic embedding. The valency of the beta carbons was satisfied by adding a hydrogen atom.

Statistical Analysis: GraphPad Prism (GraphPad Software) was employed for statistical analysis using student's t-test.

TABLE 1
CD compositional analysis from 15 μM CHAF and L-CHAF at 50 mM Na₂HPO₄ pH 4.0 buffer. Summary of
secondary structure content uses the average and standard deviation from three independent trials.

| Protein | α-helix [%] | β-sheet [%] | Random Coil [%] | −θ₂₂₂ [MRE]^{a} | −θ₂₀₈ [MRE]^{a} | θ₂₂₂/θ₂₀₈ | Tₘ [° C.] |
|---|---|---|---|---|---|---|---|
| CHAF | 13.4 ± 2.9 | 32.9 ± 13.7 | 53.7 ± 3.2 | 8,000 ± 2,000 | 7,000 ± 1,000 | 1.2 ± 0.2 | 54.9 ± 1.8 |
| L-CHAF | 91.0 ± 4.5 | 0.0 ± 0.0 | 9.0 ± 4.5 | 25,000 ± 2,000 | 25,000 ± 1,000 | 1.0 ± 0.0 | 75.8 ± 9.2 |

^{a}Mean Reside Ellipticity [10³ deg cm² dmol⁻¹]

TABLE 2
ATR-FTIR compositional analysis of 500 μM CHAF and L-CHAF
at 50 mM Na₂HPO₄ pH 4.0 buffer. Summary of secondary structure
content uses the average and standard deviation of the integrated
area of deconvoluted peaks from three independent trials.

| Protein | α-helix [%] | β-sheet [%] | Random Coil [%] |
|---|---|---|---|
| CHAF | 31.1 ± 4.9 | 37.2 ± 7.5 | 31.8 ± 2.6 |
| L-CHAF | 33.6 ± 2.3 | 43.4 ± 2.5 | 33.0 ± 1.2 |
| Denatured CHAF | 31.0 ± 3.2 | 34.2 ± 7.4 | 34.1 ± 4.7 |
| Denatured L-CHAF | 34.9 ± 0.9 | 28.7 ± 6.0 | 36.3 ± 6.9 |

TABLE 3
Computed redox potential of selected Phe and His
residues from the CHAF system. The redox potential
number is calculated with respect to SHE.

| Residue | State | VEG (eV) | ΔGₒₓ (eV) | E⁰ vs SHE (V) |
|---|---|---|---|---|
| Phenylalanine | Red | 8.761 | 7.053 | 2.613 |
| | Ox | 5.345 | | |
| Histidine | Red | 9.540 | 7.158 | 2.718 |
| | Ox | 4.776 | | |

### Example 2

This example provides a description of the methods and peptides/proteins of the present disclosure.

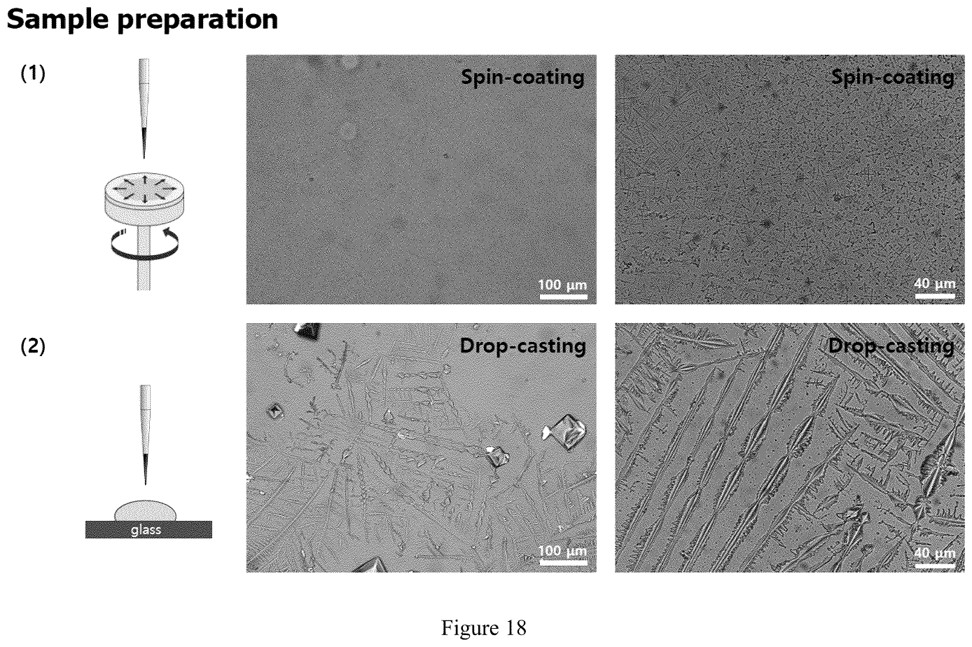

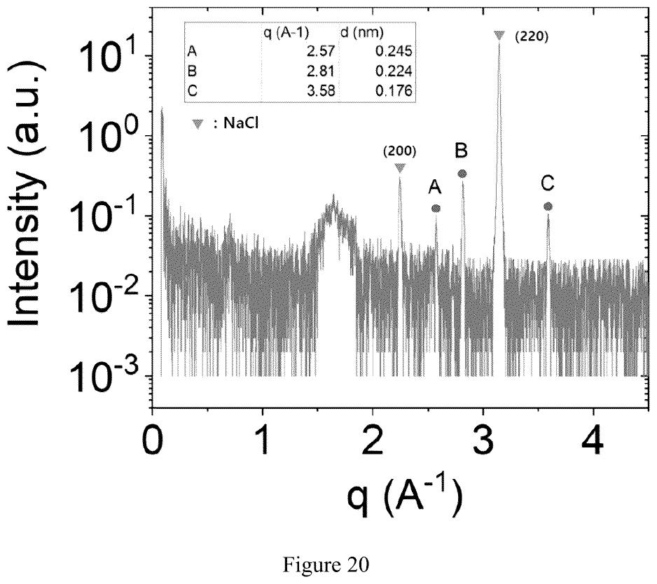

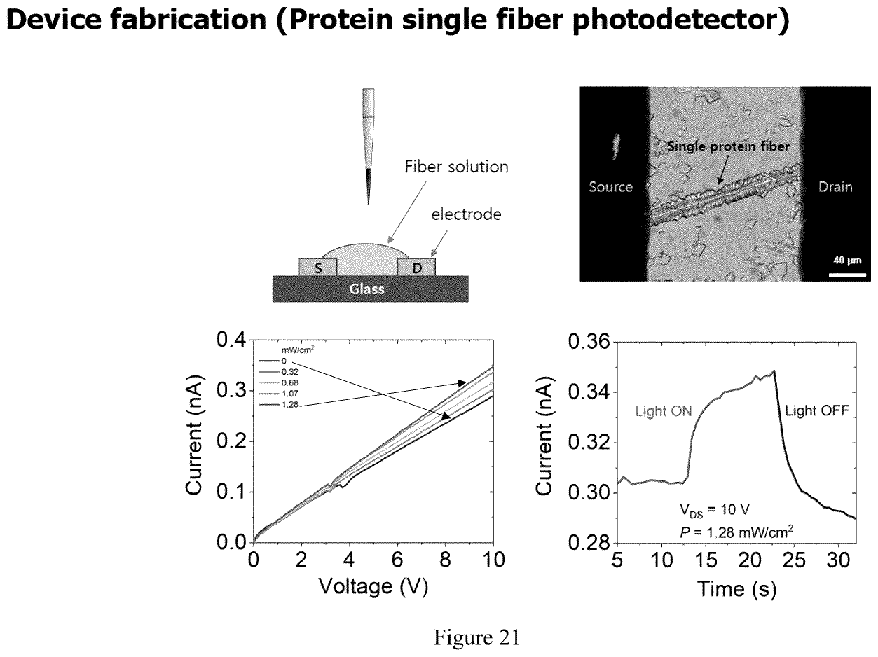

FIGS. 18 to 22 show IR responses to single protein fibers.

There are two methods for sensitizing the CF protein (FIG. 18). In the case of spin-coating, they show too low density of medium to make the CF protein, but the drop-casting method can continuously provide enough medium to form the CF protein because the drop is localized on the specific area. The 30 μL of the precursor was spin-coated on the glass substrate at 3000 rpm for 30 s and also the same amount of the precursor was dropped on the same substrate.

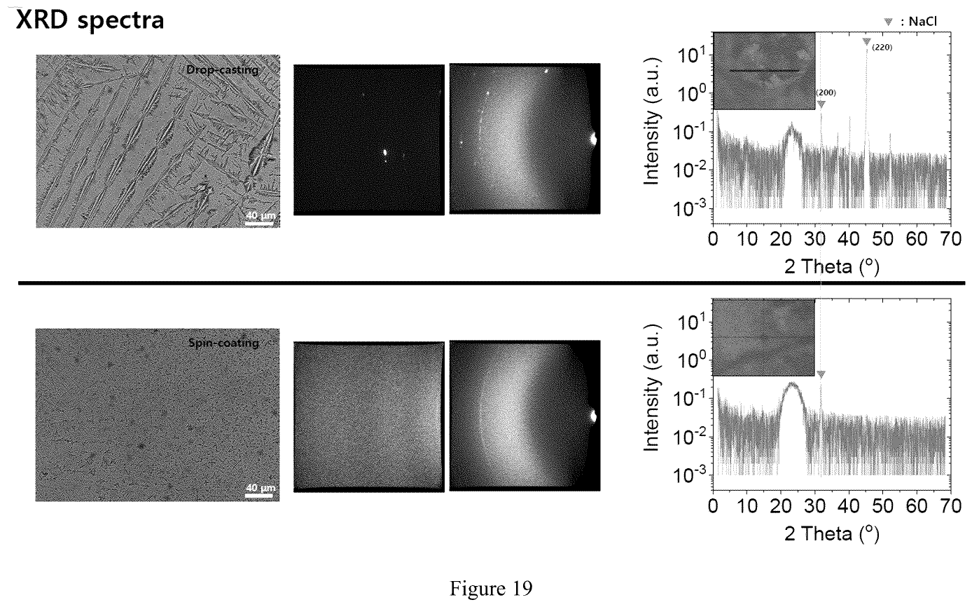

The surface morphology and elementary distribution of the perovskite thin films were investigated by X-ray Diffraction (Bruker AXS D8 Discover GADDS micro diffractometer with Cu-Ku source) (FIG. 19). The XRD spot is elliptical with an approximate average size of 800 μm. The samples were oscillated in two orthogonal directions to average the data over a 2×2 mm² area. The CF protein shows distinct specific peaks compared with a peak of NaCl which is the buffer solution.

For the CF protein PD, the dark current at 5 and 10 V was measured to be 0.15 and 0.29 nA. Upon light illumination, the current increased to a maximum value of 0.18 and 0.35 nA at 5 and 10 V under an illumination power of 1.0 mW/cm². A maximum photoinduced current of 0.35 nA was measured at an applied bias of 10 V and light power of 1.0 mW/cm². The EQE, a useful physical quantity to evaluate a PD's electrical sensitivity to light, represents the number of photoinduced carriers generated per incident photons and can be defined as, EQE=ΔI hc/(pAλe), where ΔI is the difference between the photoexcited current and the dark current, h is Planck's constant, c is the velocity of light, p is the light intensity, A is the effective irradiated area, λ is the light wavelength, and e is the electron charge.

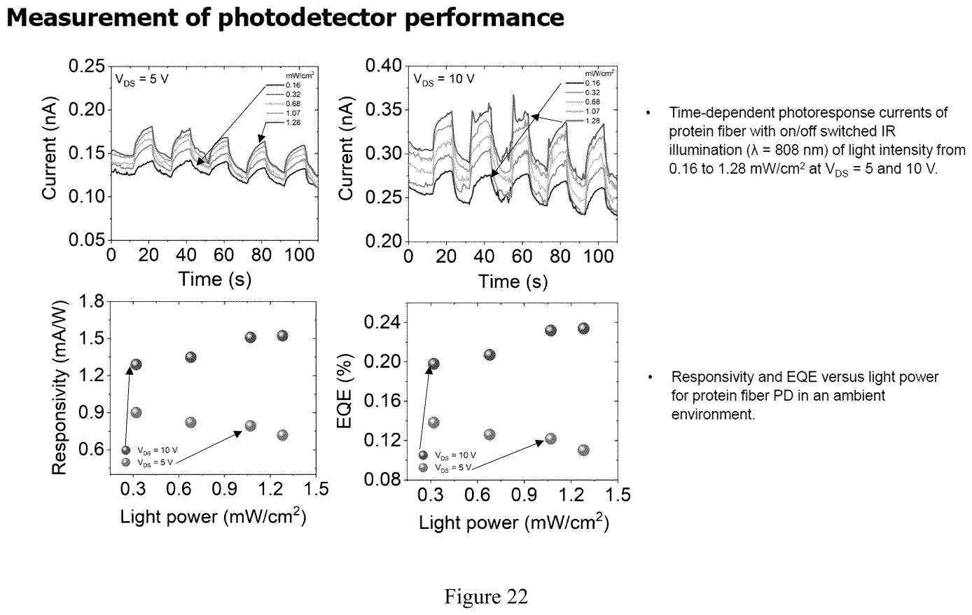

FIG. 22 displays the responsivity and EQE with respect to the incident light power at an applied bias of 5 and 10 V. Two distinct trends are apparent at the applied voltages of 5 and 10 V. EQE at the 5 V decrease with increasing light intensity due to the increased recombination rate of photoexcited carriers, while the EQE first increases with increasing light intensity.

Even though CF protein PD does not absorb the IR light, it clearly reacted with IR light. It was inferred that the IR-induced heating could make the electrical conduction of CF protein fiber to be reacted with IR light because the CF protein typically is very sensitive to thermal energy.

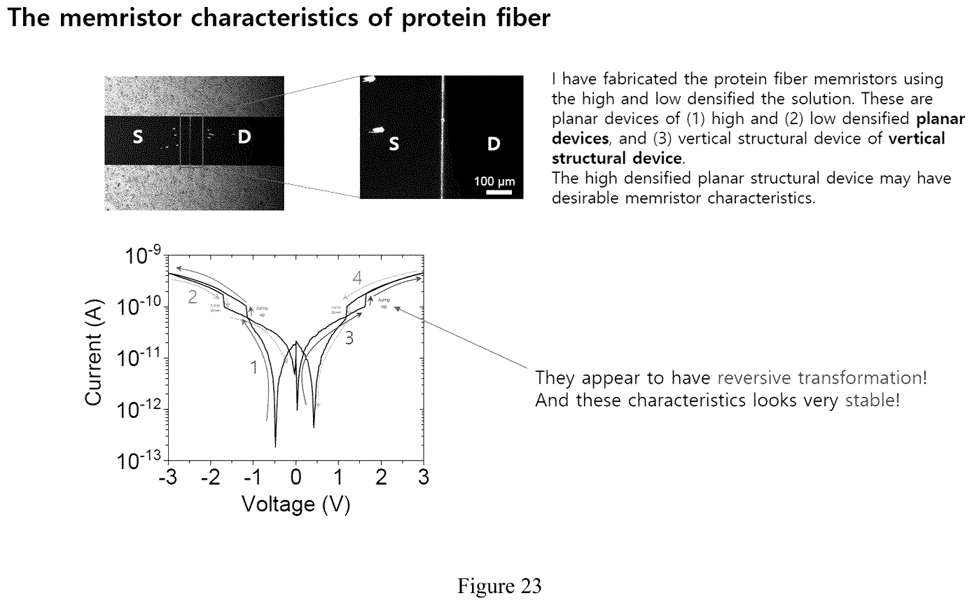

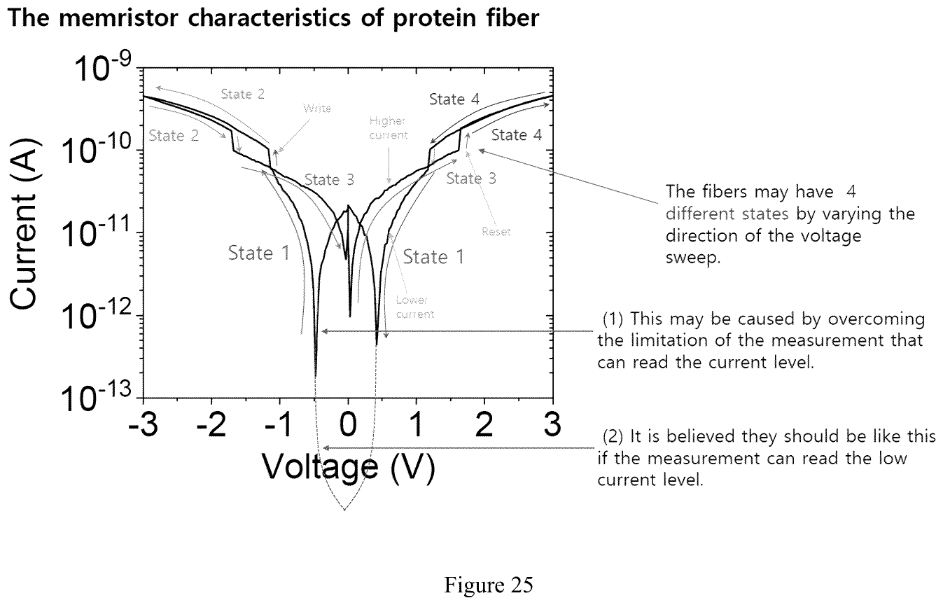

FIG. 23 to 26 show the memristor characteristics of thin films made from the proteins of the present disclosure.

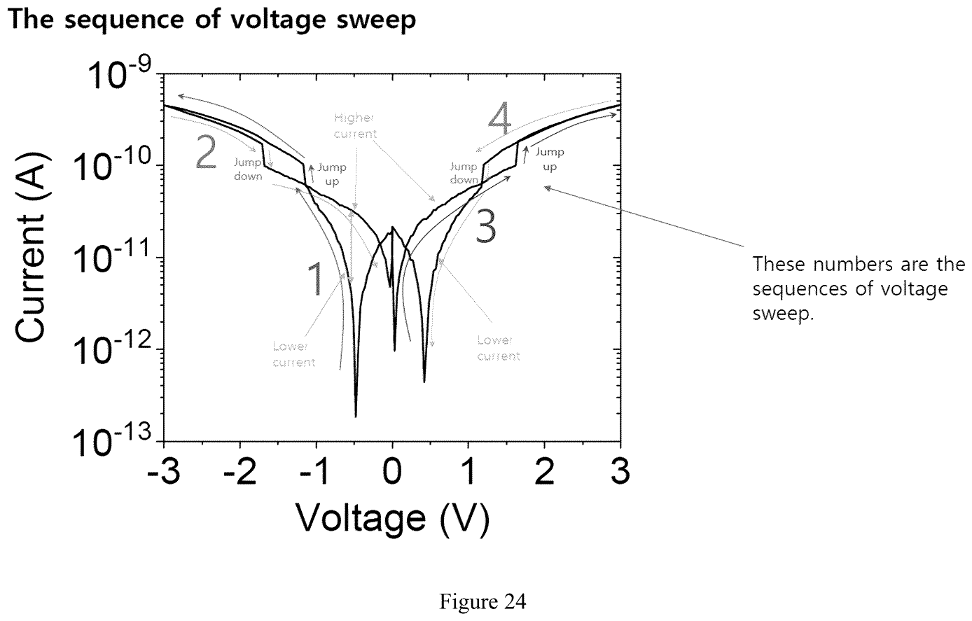

In detail, for the measurement of memristor (FIG. 24), a double sweeping of voltage was used. In the plot, the numbers are the count of sweeping. When swept firstly f, it shows some jump point in forward direction, and also in the reverse direction they showed the jump down. However, when it came back to zero voltage, they are different phase because they have very different current level between them. And also, for the positive bias, the changed phase is kept to lower, and start at the lower current state, and after they undergo the jump, they can be higher current state.

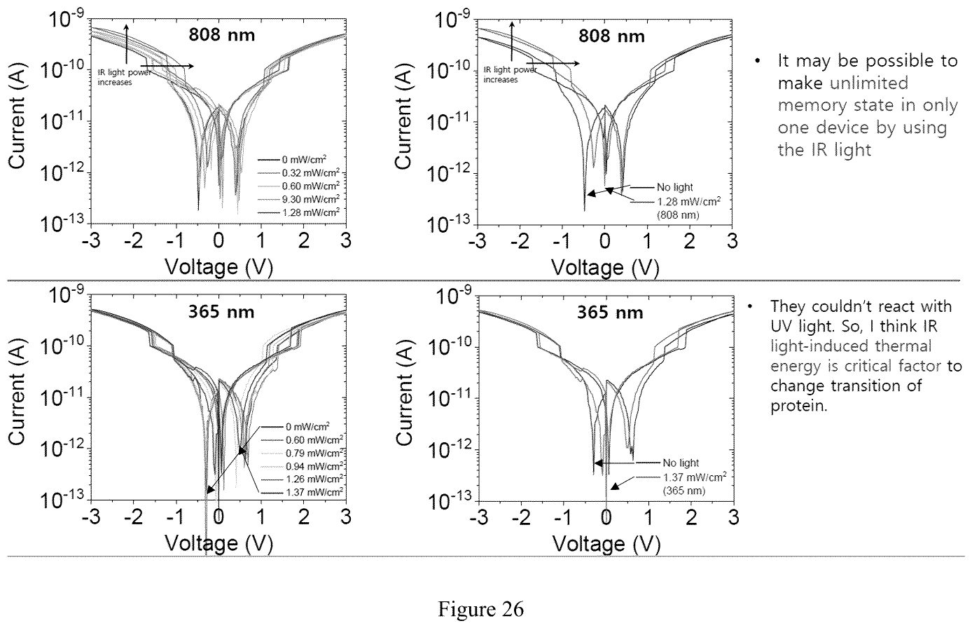

The data of FIG. 26 shows IR light dependent memristor characteristics. They can show five different lower and higher current level state as varying the IR current. It is believed that IR light induced thermal energy is a factor to change the state of them. Without intending to be bound by any particular theory, because the state to the unlimited different state can be controlled, it is considered that the memory can show an unlimited state.

Although the present disclosure has been described with respect to one or more particular embodiments and/or examples, it will be understood that other embodiments and/or examples of the present disclosure may be made without departing from the scope of the present disclosure.

1. A protein or peptide having or comprising the following sequence:
VX₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄
X₂₅X₂₆X₂₇X₂₈X₂₉X₃₀X₃₁X₃₂X₃₃X₃₄X₃₅X₃₆X₃₇,
wherein
X₁ is A, E, D, R, H, K, Q, N, or S;
X₂ is A, E, N, or Q;
X₃, X₆, X₁₀, X₁₃, X₁₇, X₂₀, X₂₄, X₂₇, X₃₁, and X₃₄ are each independently an aromatic amino acid residue chosen from F, Y, and W; a hydrophobic amino acid residue chosen from L, I, V, M, and A; or a neutral amino acid residue chosen from L, I, V, M, and A;
X₄ is A, E, R, D, H, I, L, T, K, Q, or N;
X₅ is A, F, Q, R, K, H, D, S, or E;
X₇ is A, K, or E;
X₈ is A, K, E, D, R, H, Q, or N;
X₉ is A, T, I, L, or Q;
X₁₁ is A, E, D, H, P, I, L, K, Y, N, Q, or R;
X₁₂ is A, Q, H, E, D, K, R, or N;
X₁₄ is A, L, D, E, K, or I;
X₁₅ is A, E, D, H, Y, I, L, R, K, Q, or N;
X₁₆ is A, E, or Q;
X₁₈ is A, K, E, D, K, R, H, N, or Q;
X₁₉ is A, N, D, K, R, H, Q, or E;
X₂₁ is A, N, or Q;
X₂₂ is K, A, E, I, L, M, R, H, D, Q, N, or S;
X₂₃ is A, Q, N, I, or L;
X₂₅ is A, H, Q, R, K, D, N, Y, I, E, L, or T;
X₂₆ is A, D, E, R, K, Q, H, N, or T;
X₂₈ is A, R, E, D, K, H, N, Q, or T;
X₂₉ is A, H, E, R, D, K, I, L, N, Q, T, or Y;
X₃₀ is L, A, D, K, I, N, or Q;
X₃₂ is E, D, K, H, N, Q, A, L, R, I, or Y;
X₃₃ is A, N, Q, D, E, H, K, R, or S;
X₃₅ is S, A, P, or Q;
X₃₆ is A, K, T, D, R, H, N, Q, or E; and
X₃₇ is A, L, I, K, D, N, Q, or R.

2. The protein or peptide according to claim 1, wherein X₃, X₆, X₁₀, X₁₃, X₁₇, X₂₀, X₂₄, X₂₇, X₃₁, and X₃₄ are each independently F or L.

3. The protein or peptide according to claim 2, wherein X₃, X₆, X₁₀, X₁₃, X₁₇, X₂₀, X₂₄, X₂₇, X₃₁, and X₃₄ are F.

4. The protein or peptide according to claim 1, wherein the sequence is or comprises:
```
(SEQ ID NO: 2)
VKEX₃TFX₆KNTX1₀PQX₁₃LREX₁₇QEX₂₀NAAX₂₄QDX₂₇RELX₃₁RQX₃₄SKL;
  (SEQ ID NO: 3)
VKEX₃LFX₆KKTX₁₀EQX₁₃LEEX₁₇KEX₂₀NKAX₂₄HDX₂₇RHLX₃₁ENX₃₄SKL;
  (SEQ ID NO: 4)
VKEX₃LFX₆KNTX₁₀YQX₁₃LLEX₁₇KEX₂₀NEAX₂₄YDX₂₇RHLX₃₁QQX₃₄SKL;
  (SEQ ID NO: 5)
VKEX₃TFX₆KKTX₁₀EQX₁₃AEEX₁₇KEX₂₀NKAX₂₄HDX₂₇RHAX₃₁ENX₃₄SKK;
  (SEQ ID NO: 6)
VKEX₃TFX₆KNTX₁₀EQX₁₃AEEX₁₇KEX₂₀NKAX₂₄HDX₂₇RHQX₃₁ENX₃₄SKQ;
  (SEQ ID NO: 7)
VKEX₃TFX₆KKQX₁₀EQX₁₃AEEX₁₇KEX₂₀NKAX₂₄HDX₂₇RHAX₃₁ENX₃₄SKK;
  (SEQ ID NO: 8)
VKEX₃TFX₆KKTX1₀EQX₁₃AEEX₁₇KEX₂₀NKAX₂₄HDX₂₇RHAX₃₁ENX₃₄SKK;
  (SEQ ID NO: 9)
VSEX₃TEX₆KKTX₁₀EHX₁₃AKEX₁₇KEX₂₀NKAX₂₄HTX₂₇RHAX₃₁ENX₃₄AKN;
or
(SEQ ID NO: 10)
VKEX₃KFX₆KNTX₁₀PQX₁₃LREX₁₇QNX₂₀NMAX2₄QDX_{27R}ELX₃₁QQX₃₄STL.
```

5. The protein or peptide according to claim 1, wherein the sequence is or comprises:
(SEQ ID NO: 12)
MRGSHHHHHHGSIEGRVX₁X₂X₃X₄X₅X₆X₇X₈X₉X₁₀X₁₁X₁₂X₁₃X₁₄X₁₅X₁₆X₁₇
X₁₈X₁₉X₂₀X₂₁X₂₂X₂₃X₂₄X₂₅X₂₆X₂₇X₂₈X₂₉X₃₀X₃₁X₃₂X₃₃X₃₄X₃₅X₃₆X₃₇

6. The protein or peptide according to claim 5, wherein the sequence is or comprises:
```
  (SEQ ID NO: 13)
MRGSHHHHHHGSIEGRVKEX₃TFX₆KNTX₁₀PQX₁₃LREX₁₇QEX₂₀NAAX₂₄QD
X₂₇RELX₃₁RQX₃₄SKL;
  (SEQ ID NO: 14)
MRGSHHHHHHGSIEGRVKEX₃LFX₆KKTX₁₀EQX₁₃LEEX₁₇KEX₂₀NKAX₂₄HD
X₂₇RHLX₃₁ENX₃₄SKL;
  (SEQ ID NO: 15)
MRGSHHHHHHGSIEGRVKEX₃LFX₆KNTX₁₀YQX₁₃LLEX₁₇KEX₂₀NEAX₂₄YD
X₂₇RHLX₃₁QQX₃₄SKL;
  (SEQ ID NO: 16)
MRGSHHHHHHGSIEGRVKEX₃TFX₆KKTX₁₀EQX₁₃AEEX₁₇KEX₂₀NKAX₂₄HD
X₂₇RHAX₃₁ENX₃₄SKK;
  (SEQ ID NO: 17)
MRGSHHHHHHGSIEGRVKEX₃TFX₆KNTX₁₀EQX₁₃AEEX₁₇KEX₂₀NKAX₂₄HD
X₂₇RHQX₃₁ENX₃₄SKQ;
  (SEQ ID NO: 18)
MRGSHHHHHHGSIEGRVKEX₃TFX₆KKQX₁₀EQX₁₃AEEX₁₇KEX₂₀NKAX₂₄HD
X₂₇RHAX₃₁ENX₃₄SKK;
  (SEQ ID NO: 19)
MRGSHHHHHHGSIEGRVKEX₃TFX₆KKTX₁₀EQX₁₃AEEX₁₇KEX₂₀NKAX₂₄HD
X₂₇RHAX₃₁ENX₃₄SKK;
  (SEQ ID NO: 20)
MRGSHHHHHHGSIEGRVSEX₃TEX₆KKTX₁₀EHX₁₃AKEX₁₇KEX₂₀NKAX₂₄HT
X₂₇RHAX₃₁ENX₃₄AKN;
or
(SEQ ID NO: 21)
MRGSHHHHHHGSIEGRVKEX₃KFX₆KNTX₁₀PQX₁₃LREX₁₇QNX₂₀NMAX₂₄QD
X₂₇RELX₃₁QQX₃₄STL.
```

7. A protein or peptide having or comprising the following sequence:
(SEQ ID NO: 22)
X₃₈TQX₃₉KNTX₄₀PQX₄₁LREX₄₂QNX₄₃NAAX₄₄QDX₄₅RELX₄₆QQX₄₇,
wherein X₃₈, X₃₉, X₄₀, X₄₁, X₄₂, X₄₃, X₄₄, X₄₅, X₄₆, and X₄₇ are each independently an aromatic amino acid residue chosen from F, Y, and W; a hydrophobic amino acid residue chosen from L, I, V, M, and A; or a neutral amino acid residue chosen from Q, N, T, and S.

8. The protein or peptide according to claim 7, wherein X₃₈, X₃₉, X₄₀, X₄₁, X₄₂, X₄₃, X₄₄, X₄₅, X₄₆, and X₄₇ are each independently F or L.

9. The protein or peptide according to claim 8, wherein X₃₈, X₃₉, X₄₀, X₄₁, X₄₂, X₄₃, X₄₄, X₄₅, X₄₆, and X₄₇ are F.

10. The protein or peptide according to claim 7, wherein sequence is or comprises:
MRGSHHHHHHSIEGRVKEX₃₈TQX₃₉KNTX₄₀PQX₄₁LREX₄₂QNX₄₃NAAX₄₄Q
DX₄₅RELX₄₆QQX₄₇.

11. A protein or peptide having or comprising the following sequence:
MQLX₄₈X₄₉X₅₀X₅₁LX₅₂EX₅₃QX₅₄X₅₅NAAX₅₆QX₅₇X₅₈RX₅₉LX₆₀X₆₁QX₆₂V
X₆₃EX₆₄TX₆₅X₆₆KX₆₇TX₆₈X₆₉X₇₀X₇₁DX₇₂SX₇₃X₇₄X₇₅X₇₆,
wherein,
X₄₈, X₅₁, X₅₃, X₅₅, X₅₆, X₅₈, X₆₀, X₆₂, X₆₄, X₆₆, X₆₈, X₇₁, and X₇₃ are each independently an aromatic amino acid residue chosen from F, Y, and W; a hydrophobic amino acid residue chosen from L, I, V, M, and A; or a neutral amino acid residue chosen from Q, N, T, and S;
X₄₉ is P or K;
X₅₀ is Q or H;
X₅₂ is R or K;
X₅₄ is E or K;
X₅₇ is D or S;
X₅₉ is E or T;
X₆₁ is R or Q;
X₆₃ is V or E;
X₆₅ is F or Q;
X₆₇ is D or E;
X₆₉ is M or E;
X₇₀ is E or N;
X₇₂ is A or S;
X₇₄ is K or absent;
X₇₅ is L or absent; and
X₇₆ is N or absent.

12. The protein or peptide according to claim 11, wherein X₄₈, X₅₁, X₅₃, X₅₅, X₅₆, X₅₈, X₆₀, X₆₂, X₆₄, X₆₆, X₆₈, X₇₁, and X₇₃ are each independently F or L.

13. A protein or peptide according to claim 12, wherein X₄₈, X₅₁, X₅₃, X₅₅, X₅₆, X₅₈, X₆₀, X₆₂, X₆₄, X₆₆, X₆₈, X₇₁, and X₇₃ are F.

14. A protein or peptide fiber comprising one or more protofibers comprising one or more proteins or peptides according to claim 1.

15. The protein or peptide fiber according to claim 14, wherein one or more compounds are bound to the protein or peptide fiber.

16. The protein or peptide fiber according to claim 15, wherein the one or more compounds are dyes, antibiotics, alkaloids, lipids, fatty acids, sugars, amino acids, phenolic compounds, extracellular materials, metals, nucleic acids, and combinations thereof.

17. The protein or peptide fiber according claim 14, wherein the protein or peptide fiber has a fiber diameter of about 20 nm to about 2 μm.

18. A composition comprising a plurality of protein or peptide fibers according to claim 14.

19. A device comprising a protein or peptide according to claim 1.

20. The device according to claim 19, wherein the device is a memristor.
